(19) United States
(12) Patent Application Publication
(10) Pub. No.: US 2009/0275496 A1
(43) Pub. Date: Nov. 5, 2009

(54) Effective quantitation of complex peptide mixtures in tissue samples and improved therapeutic methods

(75) Inventors: Sam Baldwin, Westford, MA (US); Dustan Bonnin, Belmont, MA (US); Keith Johnson, Hudson, MA (US); Jeff Krieger, Newtonville, MA (US); James Rasmussen, Cambridge, MA (US); Bei Yu, West Roxbury, MA (US); Eric Zanelli, Sudbury, MA (US); Jianxin Zhang, Acton, MA (US); Kathryn H. Collins, Plymouth, MA (US); Michelle Genova, Cambridge, MA (US); Joseph Kovalchin, Belmont, MA (US)

Correspondence Address: ROPES & GRAY LLP, PATENT DOCKETING 39/41, ONE INTERNATIONAL PLACE, BOSTON, MA, 02110-2624, US

(73) Assignee: Peptimmune, Inc., Cambridge, MA (US)

(21) Appl. No.: 12/316,297

(22) Filed: Dec. 10, 2008

Related U.S. Application Data
(63) Continuation-in-part of application No. 11/283,405, filed on Nov. 17, 2005.
Application No. 11/283,405 is a continuation-in-part of application No. PCT/US05/16340, filed on May 9, 2005.
Application No. PCT/US05/16340 is a continuation-in-part of application No. PCT/US05/16344, filed on May 9, 2005.
Application No. PCT/US05/16344 is a continuation-in-part of application No. 11/283,406, filed on Nov. 17, 2005.
Application No. 11/283,406 is a continuation-in-part of application No. PCT/US05/16340, filed on May 9, 2005.
Application No. PCT/US05/16340 is a continuation-in-part of application No. PCT/US05/16344, filed on May 9, 2005.
(60) Provisional application No. 61/007,091, filed on Dec. 10, 2007.
(60) Provisional application No. 60/569,292, filed on May 7, 2004.
(60) Provisional application No. 60/663,333, filed on Mar. 18, 2005.
(60) Provisional application No. 60/569,292, filed on May 7, 2004.
(60) Provisional application No. 60/663,333, filed on Mar. 18, 2005.

(51) Int. Cl. A61K 38/02; G01N 33/53; A61P 37/00

(52) U.S. Cl. 514/2; 435/7.1

(57) ABSTRACT

The instant invention provides methods for the detection and quantitation of complex peptide mixtures in tissue samples and functional readouts of the results of administration of such complex peptide mixtures. The instant invention further provides methods for administering complex peptide mixtures to a subject in need thereof, the dosage regimen and quantity determined based on the above mentioned method for detection and quantitation.

### CROSS-REFERENCE TO RELATED APPLICATIONS

This application claims priority to U.S. Provisional application No. 60/007,091 filed Dec. 10, 2007, and is a continuation-in-part of U.S. application Ser. No. 11/283,406 filed Nov. 17, 2005.

### BACKGROUND OF THE INVENTION

### Introduction

Peptide products have a wide range of uses as therapeutic and/or prophylactic reagents for prevention and treatment of disease. Many peptides are able to regulate biochemical, physiological, or immunological processes to either prevent disease or provide relief from symptoms associated with disease. For example, peptides derived from viral or bacterial proteins have been used successfully as vaccines for prevention of infectious diseases. Additionally, peptides have been successfully utilized as therapeutic agents for treatment of disease symptoms. Such peptides fall into diverse categories such as, for example, hormones, enzymes, serum proteins, cytokines, immunomodulators, or an effector or regulator of any of these functional proteins.

To manifest its proper biological and therapeutic effect, a compound must be present in appropriate concentrations for a certain amount of time at sites where its target proteins or cells reside or accumulate. To use a composition as a therapeutic, the pharmacokinetics, i.e. absorbtion, distribution, metabolism and clearance, of the active ingredient must be determined for a safe and effective dosage. In addition, in case of a therapeutic peptide, maintaining a specific three-dimensional configuration may be necessary for a biological effect. However, many peptides and proteins are labile within the body, making the determination of the pharmacokinetics problematic. Thus, investigators have developed rapid and highly sensitive analytical peptide detection systems well known in the art for use in developing dosing regiments that help investigators to maintain a steady concentration of peptide in blood circulation.

Among therapeutic peptides, immune-competent peptides have an additional functional requirement: an immune-competent peptide needs to undergo some level of cellular processing to modulate immune function through the T cell receptor:major histocompatibility complex (“MHC”). Thus, the pharmacokinetics of the peptide as administered, freely circulating in the subject's body without substantive processing, is not directly applicable to the true bioavailability or bioactivity of the immune-competent peptide. Indirect functional measurements have therefore been developed and are well known in the prior art to determine the true bioactivity, to assist investigators to determine an effective dosing regimen and increase the efficacy of such peptide regimens, and to minimize the incidence and severity of side effects.

However, the currently available methods of determining dose and dosing regimen of immunomodulatory peptide therapies are inadequate. It has been understood for some time that the antigenicity of polypeptides varies between species (Maurer, P. (1970) J. Immunol. 105:1011-12). This notion is important because treatment modalities for immunomodulatory peptides that are designed in a rodent system and then transferred to the human may not be the most effective. Combined with an inability to meaningfully utilize analytical pharmacokinetic methods, investigators have little experimentally-derived rationale to set dose and dosing regimen, short of trials in actual subject groups. This situation is particularly accurate when applied to the combined use of multiple peptides, pooled APLs, or complex peptide mixtures, where the ability to detect previously administered multiplicity of distinct peptides is an unsolved problem. There is, therefore, an unmet need to solve the problem of an inability to use predictive pharmacokinetic measurements to design more effective peptide therapeutic regimens.

Challenges in Immunomodulation

Many disease conditions are, at least in part, a result of an unwanted or excessive immune response within an organism. The rejection of a transplanted organ is an axiomatic example of an unwanted immune response. Successful transplantation depends on preventing such unwanted immune responses and allowing the graft to continually avoid the immune attacks by the subject (“sustained chimerism”). Under experimental conditions, sustained chimerism can be induced for short periods of time by peptides that are closely related to those that stimulate graft-rejecting immune responses. (Murphy et al. (2003) J. Am. Soc. Nephrol. 14:1053-1065; LeGuern (2003) Trends Immunol. 24:633-638.) However, the effect is short-lived partly due to the likelihood of “epitope spreading” (N. Suciu-Foca et al. (1998) Immunol. Rev. 164:241). Epitope spreading is a phenomenon in which, over time, the body starts recognizing areas adjacent to the original epitope as new targets of immune response. The result is a renewed attack on the graft even though the original epitopes have been accepted by the graft recipient.

To avoid graft rejection, currently, transplantation patients are often treated with immunosuppressive therapies that depress the overall immune response and reactivity in a patient. Immunosuppressive therapies attempt to attenuate the reaction of the body to an already-triggered immune response, and are accompanied by numerous undesirable side effects, including the generation of transplant-related malignancies such as Kaposi's sarcoma.

Immunomodulation, in contrast to immunosuppression, targets the onset of unwanted immune responses, and regulates immune system's activation. Immunomodulation can be attempted in a manner specific or non-specific to an antigen/epitope. An example of the non-specific treatment aims to directly control T lymphocytes or their functions using specific antibodies. Such treatment reduces but does not completely prevent unwanted immune reactions, necessitating continued use of immunosuppressants, albeit at lower doses or frequencies than when immunosuppressants are used alone. It is effective only in that it reduces side effects compared to treatment with immunosuppressive compounds only. Further, these therapies also still suffer from the unattractive side effects of compromised overall immune function.

In contrast, antigen/epitope-specific treatment aims to regulate the immune system's response against a particular antigenic determinant. Because of the specific targeting, antigen-specific immunomodulation avoids the undesirable overall immune compromise. However, epitope spreading limits the long-term effectiveness of this method, as it necessarily targets a particular epitope. (N. Suciu-Foca et al. (1998) Immunol. Rev. 164:241). Thus, in transplantation, it has been reluctantly accepted that, in the absence of the ability to modulate the relevant antigenic determinants over time, the only alternatives are non-specific immunomodulatory, or immunosuppressive therapies.

Other examples of unwanted immune responses are autoimmune diseases, caused by an inappropriate immune response directed against a self antigen (an autoantigen). Autoimmune diseases include, among others, rheumatoid arthritis (RA), multiple sclerosis (MS), human type I or insulin-dependent diabetes mellitus (IDDM), autoimmune uveitis, primary biliary cirrhosis (PBC) and celiac disease.

In the normal state of self-tolerance, T cells and B cells capable of reacting against autoantigens are prevented from being generated, or altered centrally. In autoimmune diseases, this mechanism somehow fails. The cell surface proteins that play a central role in regulation of immune responses are the MHC molecules (Rothbard, J. B. et al. (1991) Annu. Rev. Immunol. 9:527) through their ability to bind and present processed peptides to T cells. The immune reaction to a particular antigenic determinant can be intervened through either the TCR's recognition of complexes formed by MHC and antigens. In the alternative, a treatment can be through the B cell receptor's (“BCR”) recognition of the epitope itself. Compared to transplantation rejection, the offending antigenic determinant(s) is/are generally more restricted and definable in autoimmune diseases, and now known for many autoimmune diseases. MHC molecules, particularly those encoded by MHC class II genes, have a large number of allelic variants. Only a few of those many allelic forms are reactive to the disease-related antigenic determinants. Patients of an autoimmune disease, for example MS or RA, tend to carry one or more such disease-related MHC class II alleles. Thus, antigen-specific treatments are being explored, but many of the existing or prospective therapeutic agents to treat autoimmune diseases are still not specific to any particular antigenic determinant.

It has previously been shown that mixtures of related peptides may be therapeutically more effective than a single peptide. Lustgarten et al. (2006) J. Immunol. 176: 1796-1805; Quandt et al. (2003) Molec. Immunol. 40: 1075-1087. The effectiveness of a complex peptide mixture as opposed to a single peptide is based on the likelihood of interaction with the newly offending epitopes that emerged via the process of epitope spreading. (Immunol. Rev. 1998, 164:241). Therefore, the art has evolved to include multiple peptide mixtures in place of once-promising single peptide compositions that did not exhibit high therapeutic value, or those the effectiveness of which diminishes over time.

An example of such multiple peptide mixture is Copolymer-1. It achieved a moderate success to overcome the above-outlined problem. Copolymer-1 (also known as Copaxone®, glatiramer acetate, COP-1, or YEAK random copolymer) is an FDA approved, commercially available therapeutic used for the treatment of MS and one of the few therapeutics for MS that continues to be effective over time. The active component of Copolymer-1 is a random sequence polymer (RSP) composition. Random sequence polymers are mixture of amino acid polymers (bonded typically by peptide bonds) comprising two or more amino acid residues in various ratios, in a random order, which mixture is useful for invoking or attenuating certain immunological reactions when administered to a mammal. Because of the extensive diversity of the sequence mixture, a large number of therapeutically effective peptide sequences are likely included in the mixture. In addition, because of the additional peptides which may at any given time not be therapeutically effective, but may emerge as effective as the epitope shifting and spreading occurs, the therapeutic composition may remain effective over a time of dosing regimen. Random copolymers in Copolymer-1 are peptides consisting of tyrosine (Y), glutamate (E), alanine (A), and lysine (K). Copolymer-1 and other random copolymers are described, for example, in International PCT Publication Nos. WO 00/05250, WO 00/05249; WO 02/59143, WO 0027417, WO 96/32119, WO/2005/085323, in U.S. Patent Publication Nos. 2004/003888, 2002/005546, 2003/0004099, 2003/0064915 and 2002/0037848, and in U.S. Pat. Nos. 6,514,938, 5,800,808 and 5,858,964.

Despite the moderate success, Cop-1 has been shown to ameliorate MS but does not suppress the disease entirely, and is ineffective in a majority of patients (Bornstein, M. B., et al., (1987) New Engl. J. Med. 317:408; Johnson, K. P. et al. (1995) Neurology 45:1268). Another disadvantage of the current Cop-1 therapy is the amorphic compound itself, produced by solution phase synthesis definable only via molecular weight which generates lot to lot variability.

Other RSP compositions intended for therapeutic uses have been described. The work originated by Strominger et al. (WO/2003/029276) and developed further by Rasmussen et al. (US 2006/0194725) describes random copolymers consisting of the amino acids Y, F, A, and K. Compared to Copolymer-1, alanine content relative to other amino acids was increased based on Pinchuck and Maurer (J. Exp Med 122(4), 673-9, 1965), who described how an EAK polymer with higher alanine content (among the range of 10-60 mole percent) produced “better antigens”; Rasmussen et al. in fact demonstrated that a YFAK with a molar input ratio of 1:1:1:1 (“CO-23”) was not effective in eliciting a recall response as compared to a YFAK preparation with an input ratio of 1:1:10:6. In contrast, WO/2005/032482 (the '482 publication) describes building degenerate peptide sequences based on motifs lacking alanine, exemplified by [EYYK]. The motifs are used as is, or can be altered by amino acid substitutions (defined on page 10-11 of the '482 publication). Alternatively, WO/2005/074579 discloses copolymers with defined amino acids at certain positions within otherwise random sequences.

However, the random sequence polymer approach in general has drawbacks and limitations. For example, what is effective in each motif is undefined, and the composition may contain a large proportion of truly inactive peptides that lower the concentration of the active components, or worse, adversely stimulate the immune system. Additionally, these compounds are difficult to manufacture and to obtain consistency from lot-to-lot.

Investigators have been exploring other approaches to introduce some variance into a single known amino acid sequence in an attempt to create a related but more effective peptide. One such approach is creation of altered peptide ligands (APL), defined as an analog peptide which contains a small number of amino acid changes from a starting sequence such as that of a native immunogenic peptide ligand. The created peptides with altered amino acid sequences may be pooled to prepare a composition having the advantages of a heterogeneous peptide mixture. Fairchild et al., Curr. Topics Peptide & Protein Res. 2004, 6:237-44. Each APL would have a defined sequence, but the composition may be a mixture of APLs with more than one sequence.

Another approach, which aims to provide heterogeneity with a focused variation, is the creation of directed polymer sequence (DSP). DSP uses a sequence of a known sequence or epitope as a starting point. The amino acids that make up the epitope are modified via the introduction of different, related amino acids defined by a set of rules. Modifying amino acids are chosen from amino acids chemically and physically similar to the original amino acid or from amino acids found in the same position of an equivalent protein in other organisms, and incorporated into peptides by a defined amino acid molar ratio in a process similar to that for preparing RSP. The result is a mixture of related but diverse peptides. DSP compositions are described in, among others, PCT publication WO 2007/120834 and in U.S. application Ser. No. 12/288,345 by inventors of this instant invention and their colleagues.

Another aspect that needs to be improved is the mode of administration. Current treatment modalities based on repeated dosing without consideration of either the cumulative effects of the administration, or of the disease stage may limit the potential effectiveness and cause undesired side effects.

Improvements can be made by devising particular dosing regimens. U.S. Pat. No. 6,844,314 describes treatment regimens that attempt to take advantage of the vaccine-like qualities of Cop-1, in the context of the protection of damaged nerves fibers. The invention of the '314 patent bases the optimal dose on the number of damaged nerve fibers, and the regimen of administration seems to be based on factors such as the individual patient's overall health as well as age and other physical factors such as gender and weight. However, there is still a need for improved methods for the treatment of unwanted immune responses with RSPs to achieve greater effectiveness and fewer side effects, and for such methods to be adaptable for various patients' individuality.

To this end, there is a need to develop treatment regimens that are based on defining the availability of a copolymer to the immune system so that disease conditions can be modulated more effectively and universally amongst a highly heterogeneous human population. Improved modalities will be additionally useful because RSPs have the potential to be effective for the treatment of multiple autoimmune diseases (Simpson, D. et al. (2003) BioDrugs 17(3):207-10).

However, in any of these methods using a complex peptide mixture for therapeutic use, the methods to determine the effective plasma concentration of such peptide mixtures as a whole, rather than for peptides with a defined amino acid sequence, have been far from adequate because of the heterogeneity of the peptides to be detected. Determining the in vivo status of a complex peptide mixture has further significance because, depending on the route and/or frequency of administration, the same mixture can invoke primarily inflammatory (T_{H}1 type) or primarily regulatory (T_{H}2 type) responses in the subject of administration. Administration of a complex peptide composition in a less than optimal manner may trigger adverse inflammatory responses, and in fact such responses are seen in animal models and to an extent in human subjects.

Thus, there is a need for a tool for quantitative analysis of RSP and other complex peptide mixtures to assist the in vivo evaluation of such mixtures and to determine the suitable amount and means of administration for therapeutic purposes.

### SUMMARY OF THE INVENTION

The instant invention provides methods for the detection and quantitation of complex peptide mixtures, such mixtures comprising individually-defined peptides and peptide analogs or a collection of peptides defined by certain synthesis rules and/or composition characteristics, including pools of altered peptide ligands (APL), peptide libraries, and random sequence polymer (RSP) compositions. The instant invention further provides methods for administering complex peptide mixtures to a subject in need thereof, the dosage regimen and quantity determined based on the above mentioned method for detection and quantitation.

An aspect of the invention is a means to determine biologically available quantity or concentration in vivo of administered peptide mixtures. A method of the instant invention is to detect the presence of complex peptide mixtures in subject tissue, said subject tissue having previously been in contact with the complex peptide mixture, wherein the method is carried out one or more times immediately after such contact, or within or at about 10, 20, 30, 45 minutes, 1, 2, 4, 6, 12, 24, 36, 48 hours, 3, 4, 5, 6, 7, 10 days, 2, 3, 4, 6, 8, or 12 weeks after such contact. A particular method of the instant invention is to detect the presence of complex peptide mixtures in the serum or plasma of a mammal, said mammal having been previously administered said complex mixture prior to carrying out said method within a time period described above. In certain embodiments, said mammal is a rodent. In other particular embodiments, said mammal is a human.

In certain embodiments, the method comprises immunologic detection methods. In particular, the method comprises Direct Competitive Enzyme-Linked Immunosorbent Assay (ELISA), Western blot, immunoflow cytometric detection, radioimmunoassay (RIA), or any other immunologic detection method that allows quantitative detection of specific antigens.

Another aspect of the instant invention is to provide methods of administering therapeutic and safe amounts of complex peptide mixtures to a mammalian subject, such amount based on the bioavailable portion of the dosed amount as determined by the method of quantitative detection described herein. In certain embodiments, the method further comprises the steps of including a control sample, performing a pharmacodynamic test to determine changes of physiological markers, such as hormones, enzymes, serum proteins, cytokines, immunomodulators, or an effector or regulator of any of these functional proteins, between the control sample and test samples by comparing the two results, and determining the dosage effective to induce the desired changes in pharmacodynamic parameter. In another embodiment, behavioral changes, subjective changes as reported by a subject such as amelioration of pain or a symptom of a disease, or other evidence of indirect effects are observed. In one embodiment, said mammalian subject is a rodent. In particular embodiments, the subject is mouse. In other particular embodiments, the subject is rat. In another embodiment, said subject is human.

A further aspect of the instant invention is to provide methods to predict a therapeutically optimal amount of complex mixture to be delivered to a therapeutic subject (particularly a human subject) based on data obtained from experimental subjects. Such method comprises the steps of administering therapeutically optimal amounts of complex peptide mixtures to a non-human experimental mammalian subject, determining the bioavailable portion of the dosed amount using the method of quantitative detection described herein, determining functional read outs, and predicting a therapeutically optimal amount of complex mixture to be delivered to the therapeutic subject based on the data obtained for the experimental mammalian subject and established correlation between the therapeutic and experimental subjects. In particular embodiments, the experimental subject is a rodent. In particular embodiments, the experimental subject is mouse. In other particular embodiments, the experimental subject is rat.

Yet another aspect of the instant invention is to provide an efficient and effective methods of treating a patient by administering a complex peptide mixture, such methods comprising the steps of: synthesizing peptides consisting of a complex peptide mixture by peptide synthesis, preparing a pharmaceutically acceptable formulation of said complex peptide mixture, administering said complex peptide mixture to a subject, obtaining a tissue sample from said subject, determining the amounts and/or concentrations of the complex peptide mixture in said tissue sample, determining a functional readout, correlating the said amounts of the complex peptide mixture to the functional readout, and optimizing the dosage of said complex peptide mixture to the subject by attaining the optimal functional readout. For the purposes of the instant invention, a “functional readout” is a phenotype or function of the subject, a phenotype or function of cellular material derived from the subject, or the composition of fluids derived from the subject. A functional readout also includes biosynthetic or metabolic compositions such as hormones, enzymes, serum proteins, cytokines, chemokines, growth factors, immunomodulators, and an effector or regulator of said functional readouts, In a particular embodiment, the detection step is repeated after certain time intervals to determine the time-course of bioavailability after administration. In certain embodiment, a half-life of the complex peptide mixture as a group is determined from such time course.

Another aspect of the invention is a method of improving the manufacturing process of a composition comprising a complex peptide mixture, such method comprising preparing a complex peptide mixture according to a protocol, further preparing a composition comprising said complex peptide mixture, determining the bioavailable amount of complex peptide mixture in said composition by detecting the level or degree of functional readout, comparing such readout against a standard, and adjusting the protocol or the step of preparing the composition to obtain a desired bioavailability.

### BRIEF DESCRIPTION OF THE DRAWINGS

FIG. 1 shows experimentally prepared standard curves of Copaxone (COP-1; circle) and PI-2301 RSP (square) detection by ELISA.

FIG. 2 shows the concentration of PI-2301 (Panel A) and Copaxone (Panel B) over two-hour period in the serum of CD-1 mice dosed once subcutaneously. Panel C is an additional data of PI-2301 concentration administered at a higher dose. Panel D shows a linear correlation between an administered subcutaneous dose of PI-2301 and serum concentration of PI-2301 at 30 min in CD-1 mice.

FIG. 3 shows the linear correlation between dose in mg/kg and extrapolated Cmax over two-hour period in the serum of CD-1 mice dosed once subcutaneously.

FIG. 4 shows the linear correlation between dose in mg/kg and total exposure over two-hour period in the serum of CD-1 mice dosed once subcutaneously.

FIG. 5 shows the levels of TNFα (panel A), IL-6 (panel B), CXCL1 (panel C) and CXCL2 (panel D) in the serum over time in PI-2301- and Copaxone-treated myeloid cells.

FIG. 6A shows the levels of CCL22 and FIG. 6B shows the levels of CXCL13 in the serum over time in PI-2301-treated and Copaxone-treated mice.

FIG. 7 shows the time dependent concentration change of Copaxone and PI-2301 following a single subcutaneous administration of 25 or 80 mg/kg in mice. Panel A is the time course. Panel B is a table of constants calculated from the time course.

FIG. 8 shows the bioavailability of Copaxone (Panel A) following a single subcutaneous or intravenous administration and PI-2301 (Panel B) following a single subcutaneous and intramuscular administration in mice.

FIG. 9 shows the correlation between serum concentration of PI-2301 over 15 μg/mL and death in CD1 mice.

FIG. 10 shows the titration curve of anti-PI-2301 antibody ELISA in multiple species of animals.

FIG. 11 shows the serum concentration of PI-2301 in cynomolgus monkeys after a single administration of PI-2301 intravenously or subcutaneously.

FIG. 12 shows the serum concentration of PI-2301 in rabbits after a single subcutaneous administration of PI-2301. Panel A shows subcutaneous administration at doses 20, 40, 80, 120, or 160 mg/kg and panel B shows intravenous administration at 1 mg/kg and subcutaneous administration at 2 or 10 mg/kg. Panel C shows the linear correlation between dosed amount and exposure shown by the area under the curve.

FIG. 13 shows the serum concentration of PI-2301 in rats after a single subcutaneous administration of PI-2301. Panel A shows subcutaneous administration at doses 15 or 40 mg/kg, and Panel B shows the linear correlation between dosed amount and exposure shown by the area under the curve.

FIG. 14 shows the serum concentration of PI-2301 in human male after a single subcutaneous administration at 10, 30, or 60 mg per dose.

FIG. 15 shows an inverse correlation between plasma levels of IL-1 receptor antagonist and disease score of EAE in mice.

FIG. 16 shows the effect of PI-2301 on the disease score of EAE.

FIG. 17 shows plasma levels of IL1ra after a single subcutaneous administration of PI-2301 in healthy human males.

### DETAILED DESCRIPTION OF THE INVENTION

The instant invention provides methods for the detection and quantitation of complex peptide mixtures, such mixtures comprising individually-defined peptides and peptide analogs or a collection of peptides defined by certain synthesis rules and/or composition characteristics, including pools of APLs, peptide libraries, random sequence polymer (RSP) compositions. The instant invention further provides methods for administering complex peptide mixtures to a subject in need thereof, the dosage regimen and quantity determined based on the above mentioned method for detection and quantitation.

Peptide Detection and Quantitation

Examples and preparation of the complex peptide mixtures are described below. One aspect of the instant invention is the detection of the quantity of components of a complex peptide mixture in subject tissue. An embodiment of this aspect is a method for detecting the presence of complex peptide mixture in a subject tissue, said subject tissue having previously been in contact with the complex peptide mixture, comprising the steps of obtaining a sample of said subject tissue, contacting said sample with a means to specifically detect the presence of components of said complex peptide mixture, and quantitatively detecting such presence, wherein the method is carried out one or more times immediately after such contact, or within 10, 20, 30, 45 minutes, 1, 2, 4, 6, 12, 24, 36, 48 hours, 3, 4, 5, 6, 7, 10 days, 2, 3, 4, 6, 8, 12 weeks after such contact. A particular method of the instant invention is to detect the presence of a complex peptide mixture in the serum or plasma of a mammal, said mammal having been administered said complex mixture prior to carrying out said method within a time period described above. Preparation of sera and plasma from a biological sample such as blood is well-known in the art. In a more particular method of the instant invention, said mammal is a rodent. Rodent includes but is not limited to mouse, rat, rabbit, guinea pig, and hamster. In other embodiments, said mammal is a dog, a mini-pig or other micro swine, a ferret, a primate, a cat, a sheep, a goat, or a horse. In another particular method, said primate is a cynomolgus monkey, a rhesus monkey, or a human.

In certain embodiments, the method comprises immunologic detection methods. In particular, the method comprises Direct Competitive Enzyme-Linked Immunosorbent Assay (ELISA), Western blot, immunoflow cytometric detection, radioimmunoassay (RIA), or any immunologic detection method known in the art that allows quantitative detection of specific antigens. To carry out the immunological detection methods, antibodies are prepared against a complex peptide mixture of interest by immunizing an animal with said complex peptide mixture. Methods for preparation of antibodies are well known in the art. For example, see Current Protocols in Immunology, Coligan et al., John Wiley & Sons, Inc., 2002. For the practice of instant invention, polyclonal antibodies prepared against a batch of complex peptide mixture is useful. Polyclonal antibodies are raised against a complex peptide mixture by immunizing chicken or mammals such as rabbits with the peptide mixture. The peptide mixture may be used as immunogen as is, or when appropriate, may be conjugated to a carrier protein or adsorbed onto suitable supporting matrix. Suitable carrier molecules include, but are not limited to, bovine serum albumin, thyroglobulin, ovalbumin, tetanus toxoid, and keyhole limpet hemocyanin. Methods of conjugating peptides to carrier proteins are also well known in the art and commercial kits are available for easy manipulation. Suitable supporting matrix include, but are not limited to, alum, carboxymethylcellulose, insoluble acetylated bovine serum albumin, or certain inactivated such as a rough strain of Pneumococcus. For exemplary methods of adsorption, see McDonald et al. (1972) J. Immunol., 108: 1690-1697.

To enhance immunogenicity, peptides or their conjugates are mixed with one or more adjuvants before injection. Examples of adjuvants include, but are not limited to, aluminum hydroxide, Freund's adjuvant (complete or incomplete), and immune-stimulating complexes (ISCOMs). ISCOMs can be made according to the method described by Morein, B. et al. (1984) Nature 308:457-460. Briefly, antibody-producing animals are immunized by standard amounts of the adjuvant containing mixtures and antibody titers are measured starting from two weeks after injection. One or two booster shots are given to increase the titer. When the adequate titer level is achieved, commonly after two or three months from the first immunization, immunogenic serum is collected, in case of small animals by sacrificing them and with larger animals by tapping the blood and isolating the serum. Standard curves are created using a detection method of choice.

The methods of present invention comprise any quantitative immunodetection assays convenient for the practitioner. In one embodiment, enzyme-linked immunosorbent assay (ELISA) is used. ELISA is a method well known in the art. Briefly, the bottom of 96-well microtiter plates are coated either by the antigen to be detected or by antibodies specific to the antigen detected. Ligands that specifically bind to the coated materials are added to the wells, and after a time, the wells are gently rinsed to remove excess and non-specific binding. Then a second antibody that specifically binds to the ligands are added to the wells and the wells are rinsed. The second antibody is engineered for easy detection, such as having been conjugated to radioisotope or color-producing or immunofluorescence-producing moiety.

Western blot is also well-known in the art. Briefly, samples containing target antigen are resolved by electrophoresis across polyacrylamide gel with appropriate buffer solution. The separated materials, most often proteins and peptides, are then electrophoretically transferred and adsorbed to a filter material such as PVDF. The filter material is blocked to prevent further adsorption by nonspecific proteins, peptides, and other biological materials, incubated with ligands specific to certain of the samples, and the binding is detected in a manner similar to the ELISA described above.

Complex Peptide Mixtures

Complex peptide mixtures for the purposes of describing the instant invention may include pools of APLs, peptide libraries, RSP compositions, and a limited diversity pool of peptides created by using pathogen-created variation in an epitope sequence useful in vaccinations (U.S. Pat. No. 7,118,874).

In some embodiments, RSPs which may be used in the invention include those described in International PCT Publication Nos. WO 00/05250, WO 00/05249; WO 02/59143, WO 0027417, WO 96/32119, in U.S. Patent Publication Nos. 2004/003888, 2002/005546, 2003/0004099, 2003/0064915 and 2002/0037848, in U.S. Pat. Nos. 6,514,938, 5,800,808 and 5,858,964, and those described in PCT application PCT/US05/06822. These references describe methods of synthesizing RSPs, compositions comprising RSPs, therapeutic formulations of RSPs, methods of administering RSP compositions to a subject, diseases that may be treated with RSPs, and additional therapeutically effective agents which may be co-administered to a subject in with the RSPs. The teachings of all these patents, applications and publications are herein incorporated by reference in their entirety.

In certain embodiments of the invention, the RSP composition is selected from the group consisting of Cop-1 (YEAK), YFAK, VYAK, VWAK, VEAK and FEAK. Y, F, A, K, V, W, etc. are one-letter code of amino acids that consist an RSP, and the molar input ratio of such amino acids is defined for each RSP composition. In one embodiment, the RSP is Cop-1. In another embodiment, the RSP is YFAK. In another embodiment, the RSP is a terpolymer, such as one selected from the group consisting of YAK, YEK, KEA and YEA.

“Molar input ratio” means the molar ratio of amino acids that are used to synthesize the RSP. For example, if an RSP is said to have a molar input ratio of 1:1:10:6 of Y:F:A:K, then when synthesizing by solid phase synthesis, for each cycle of elongation, a mixture of protected amino acids Y, F, A, and K in the molar ratio of 1:1:10:6 is reacted to elongate the peptide chain. On the other hand, “Molar output ratio” means the molar ratio of amino acids that actually consists the RSP peptides after synthesis. Molar output ratio is determined by analyzing the amino acid content of an RSP composition. Input and output ratios are not identical due to differences among amino acids in incorporation efficiencies.

In certain other embodiments, the method of invention comprises RSPs having certain characteristics of APLs based on known epitopes associated with diseases.

One class of RSP for which the instant invention is useful comprises the characteristics of a compilation of a multiplicity of cross-reactive T cell epitopes, conferring to the RSP the potential to functionally interact with thousands, preferably hundreds of thousands, more preferably millions, of T cell epitopes via presentation by MHC molecules, preferably MHC class II molecules. Another class of RSP is specific to T cells which may secrete soluble mediators, such as cytokines.

RSP for which the instant invention is useful may be given specific amino acid sequence characteristics such that the selected sub-group of amino acids preferentially interacts with specific T cell epitopes, some of which may be directly associated with pathogenic disorders. Preferably, such RSP comprises between two and eight kinds of amino acids connected in a random order and preferentially interact with specific T cell epitopes, some of which are or thought to be directly associated with pathogenic disorders that are exacerbated by aberrant production of soluble mediators, such as cytokines. In certain embodiments, such pathogenic disorders are linked to specific MHC class II alleles such as HLA-DR, or HLA-DQ. In particular embodiments, such RSP composition comprises polymers consisting of three, four, or five kinds of amino acids randomly connected, preferably via peptide bonds.

RSP for which the instant invention is useful may comprise a suitable quantity of an amino acid of positive electrical charge, such as lysine or arginine, in combination with an amino acid with a negative electrical charge (preferably in a lesser quantity), such as glutamic acid or aspartic acid, optionally in combination with an electrically neutral amino acid such as alanine or glycine, serving as a filler, and optionally with an amino acid adapted to confer on the copolymer immunogenic properties, such as an aromatic amino acid like tyrosine or tryptophan. Such compositions may include any of those disclosed in WO 00/005250, the entire contents of which being incorporated herein by reference.

In one embodiment of the invention, RSP contains four different amino acids, each from a different one of the following groups: (a) lysine and arginine; (b) glutamic acid and aspartic acid; (c) alanine and glycine; (d) tyrosine and tryptophan.

A specific RSP according to this embodiment of the present invention comprises in combination L-alanine (A), L-glutamic acid (E), L-lysine (K), and L-tyrosine (Y), and has a net overall positive electrical charge. One particular example is Copolymer 1 (Cop-1) also referred to as YEAK or glatiramer acetate. Cop-1 has been approved in several countries for the treatment of multiple sclerosis (MS) under the trade name, COPAXONE™ (trademark of Teva Pharmaceuticals Ltd., Petah Tikva, Israel). Cop-1 binds with high affinity and in a peptide-specific manner to purified MS-associated HLA-DR2 (DRB1*1501) and rheumatoid arthritis (RA)-associated HLA-DR1 (DRB1*0101) or HLA-DR4 (DRB1*0401) molecules. Since Cop-1 is a mixture of random polypeptides, it may contain different sequences that bind to different MHC proteins; in this case only a fraction out of the whole mixture would be an “active component.” Alternatively, the whole mixture may be competent, i.e. all polypeptides binding to any HLA-DR molecule, but this has not been shown. A Cop-1 RSP of interest has a molecular weight of about 2,000 to about 40,000 daltons, and more particularly from about 2,000 to about 13,000 daltons. Cop-1 has an average molecular weight about 4,700 to about 13,000 daltons, but includes smaller and larger peptides as well. The average molecular weight of most interest for Cop-1 is between about 5,000 and about 9,000 daltons. Thus, the Cop-1 RSP may be a polypeptide from about 15 to about 100 amino acid residues, preferably from about 40 to about 80, amino acid residues in length. In a particular embodiment, the length of Cop-1 RSP is between 35 and 75 amino acids residues. More particularly, the length of Cop-1 RSP is between 35 and 65 amino acid residues. In a particular embodiment the length of Cop-1 is about 50 amino acids. In another particular embodiment, the length of Cop-1 RSP is about 52 amino acids. In certain embodiments, Cop-1 has an average molar output ratio of about 1.0:2.0:6.0:5.0 for Y:E:A:K respectively, synthesized by solid phase chemistry well known in the art. Instead of using a single kind of amino acid for any given cycle, however, the synthesis of Cop-1 is carried out by adding a mixture of appropriately protected Y, E, A, and K at a defined ratio for each cycle. The variability in the output ratios comprises a range of about 10% between the different amino acids. Molecular weight ranges and processes for making a preferred form of Cop-1 are described in U.S. Pat. No. 5,800,808, the contents of which are hereby incorporated in the entirety.

In an embodiment of Cop-1 RSP of about 52 amino acid residues, the ratio of alanine composition in amino acid positions 31-52 is greater than in amino acid positions 11-30, and the ratio of alanine composition in amino acid positions 11-30 is greater than in amino acid positions 1-10. In a particular embodiment, residues 1-10 of the Cop-1 RSP sequence has a molar output ratio of about 1.0:2.0:5.5:5.0, residues 11-30 have a molar output ratio of about 1.0:2.0:6.0:5.0, and residues 31-52 have a molar output ratio of about 1.0:2.0:6.5:5.0, all ratios indicated for molar ratio among Y, E, A, K in that order.

For the purpose of the present invention, the phrase “Cop 1 or a Cop I-related peptide or polypeptide” is intended to include any peptide or polypeptide, that cross-reacts functionally with myelin basic protein (MBP) and is able to compete with MBP on the MHC class II in the antigen presentation. The activity of Cop-1 for the utilities disclosed herein is expected to remain if one or more of the following substitutions is made: aspartic acid (D) for glutamic acid (E), glycine (G) for alanine (A), arginine (R) for lysine (K), and tryptophan (W) for tyrosine (Y).

In another embodiment, the RSP composition contains three different amino acids each from a different one of three groups of the above mentioned groups (a) to (d). These copolymers are herein referred to as “terpolymers.” The average molecular weight is between 2,000 to about 40,000 daltons, and preferably between about 3,000 to about 35,000 daltons. In a more particular embodiment, the average molecular weight is about 5,000 to about 25,000 daltons. Exemplary terpolymers are shown in the table below. The average molar fraction of the amino acids in these terpolymers can vary and are shown in the general range.

TABLE A
Terpolymers suitable for the use in the present invention

| Amino acid | | Particular | Reference and |
|---|---|---|---|
| composition | Molar fraction range (output) | embodiment ratio | notes |
| tyrosine, | Y: about 0.005 to about 0.250 | Y: about 0.10 | Fridkis-Hareli M., |
| alanine, and | A: about 0.3 to about 0.6 | A: about 0.54 | Hum |
| lysine, (“YAK”) | K: about 0.1 to about 0.5 | K: about 0.35 | Immunol. 2000; |
| | | | 61(7): 640-50. |
| tyrosine, | Y: about 0.005 to about 0.250 | Y: about 0.26 | Variations: |
| glutamic acid, | E: about 0.005 to about 0.300 | E: about 0.16 | Y -> W; |
| and lysine | K: about 0.3 to about 0.7 | K: about 0.58 | E -> D; and/or |
| (“YEK”) | | | K -> R. |
| lysine, | K: about 0.2 to about 0.7 | K: about 0.36 | |
| glutamic acid, | E: about 0.005 to about 0.300 | E: about 0.15 | |
| and alanine | A: about 0.005 to about 0.600 | A: about 0.48 | |
| (“KEA”) | | | |
| tyrosine, | Y: about 0.005 to about 0.250 | Y: about 0.21 | Variations: |
| glutamic acid, | E: about 0.005 to about 0.300 | E: about 0.14 | Y -> W; |
| and alanine, | A: about 0.005 to about 0.800 | A: about 0.65 | E -> D; and/or |
| (“YEA”) | | | A -> G. |
| For reference: | | Y: about 0.10 | |
| tyrosine, | | E: about 0.14 | |
| glutamic acid, | | A: about 0.43 | |
| alanine, | | K: about 0.34 | |
| lysine, | | | |
| (“YEAK; Cop- | | | |
| 1”) | | | |

In a more particular embodiment, the molar fraction of amino acids of the terpolymers is about what is preferred for Cop-1. The mole fraction of amino acids in Cop-1 is glutamic acid about 0.14, alanine about 0.43, tyrosine about 0.10, and lysine about 0.34.

Another particular RSP according to this embodiment of the present invention comprises in combination L-alanine (A), L-phenylalanine (F), L-lysine (K), and L-tyrosine (Y), and herein referred to as YFAK. The length of any of such RSP is between about 25 and 300 amino acid residues. YFAK RSP that is preferred for the use in a therapeutic composition is between 35 and 75 amino acids residues. More preferably, the length of the RSP is between 35 and 65 amino acid residues. A preferred RSP has the length of is about 50 or 52 amino acids.

A particular composition of YFAK (L-tyrosine, L-phenylalanine, L-alanine and L-lysine) has a molar output ratio of about 1.0:1.2: X_{A}: 6.0 respectively, wherein X_{A} is greater than 11.0 and less than 30.0, and more particularly, greater than 20.0 and less than 30.0, and the variability in the output ratios comprises a range of about 10% between the different amino acids. The molar output ratios of YFAK of random copolymers preferred for therapeutic use are shown in Table B below:

TABLE B
Amino Acid Composition Ratios of YFAK RSP

| Y | F | A | K |
|---|---|---|---|
| 1.0: | 1.2: | 11.0 < 30.0: | 6.0 |
| 1.0: | 1.2: | 18.0: | 4.0 |
| 1.0: | 1.2: | 18.0: | 5.0 |
| 1.0: | 1.2: | 18.0: | 6.0 |
| 1.0: | 1.2: | 18.0: | 7.0 |
| 1.0: | 1.2: | 18.0: | 8.0 |
| 1.0: | 1.2: | 20.0: | 4.0 |
| 1.0: | 1.2: | 20.0: | 5.0 |
| 1.0: | 1.2: | 20.0: | 6.0 |
| 1.0: | 1.2: | 20.0: | 7.0 |
| 1.0: | 1.2: | 20.0: | 8.0 |
| 1.0: | 1.2: | 20.0 < 30.0: | 6.0 |
| 1.0: | 1.2: | 22.0: | 6.0 |
| 1.0: | 1.2: | 24.0: | 6.0 |
| 1.0: | 1.2: | 26.0: | 6.0 |
| 1.0: | 1.2: | 28.0: | 6.0 |
| 1.0: | 1.2: | 30.0: | 6.0 |

| | | |
|---|---|---|
| (Y + F = 2.2): | 18.0: | 6.0 |

| | | | |
|---|---|---|---|
| 1.0: | 1.3: | 24.0: | 6.0 |
| 0.66: | 1.54: | 18.0: | 6.0 |
| 0.88: | 1.32: | 18.0: | 6.0 |

A particular YFAK composition has an average molar output ratio of about 1.0:1.3:24.0:6.0 (Y, F, A, K respectively), prepared by solid phase synthesis known in the art.

Another YFAK composition that is preferred for therapeutic use has an average molar output ratio of YFAK is about 1.0:1.2: X_{A}:6.0, wherein X_{A} is greater than 20.0, and the ratio of alanine increases with the length of copolymer. In a particular composition, the length of such RSP is about 52 amino acid residues, and the ratio of alanine composition in amino acid positions 31-52 is greater than in amino acid positions 11-30, and the ratio of alanine composition in amino acid positions 11-30 is greater than in amino acid positions 1-10.

In one embodiment, the RSP composition used in the methods described herein are capable of binding to an MHC class II protein which, preferably, is associated with an autoimmune disease. There are at least three types of Class II MHC molecules in human: HLA-DR, HLA-DQ, and HLA-DP molecules. There are also numerous alleles encoding each type of these HLA molecules. The Class II MHC molecules are expressed predominantly on the surfaces of B lymphocytes and antigen presenting cells such as macrophages. The Class II MHC protein consists of approximately equal-sized α and β subunits, both of which are transmembrane proteins. A peptide-binding cleft is formed by parts of the amino termini of both α and β subunits. This peptide-binding cleft is the site of presentation of the antigen to T cells. Any available method can be used to ascertain whether the copolymer binds to one or more MHC class II proteins. For example, the polypeptide can be labeled with a reporter molecule (such as a radionuclide or biotin), mixed with a crude or pure preparation of MHC class II protein and binding is detected if the reporter molecule adheres to the MHC class II protein after removal of the unbound polypeptide.

In another embodiment, the RSP composition used in the methods described herein are capable of binding to an MHC class II protein associated with multiple sclerosis (MS). A polypeptide of this embodiment can have similar or greater affinity for the antigen binding groove of an MHC class II protein associated with multiple sclerosis than does Cop-1. Hence, the contemplated polypeptide can inhibit binding of or displace the binding of myelin autoantigens from the MHC class II protein. One MHC class II protein associated with multiple sclerosis is HLA-DR2 (DRB1*1501).

In another embodiment, the RSP composition used in the methods described herein are capable of binding to an MHC class II protein associated with an arthritic condition, for example, rheumatoid arthritis (RA) or osteoarthritis (OA). RSP of this embodiment can have a greater affinity for the antigen binding groove of an MHC class II protein associated with the autoimmune disease than does a type II collagen 261-273 peptide. Hence, the RSP described herein such as YFAK can inhibit binding of or displace the type II collagen 261-273 peptide from the antigen binding groove of an MHC class II protein.

In certain particular embodiments, the RSPs bind to HLA-DQA 1 molecules, and in even more preferably to one or more of HLA molecules encoded in the alleles DQA1*0501-DQB1*0201, DQA1*0301, DQB1*0401, and DQA1*03-DQB1*0302.

In other embodiments, the RSPs bind to certain HLA-DQ molecules that predispose the carrier of such molecules to autoimmune-associated diseases, such as type I diabetes and celiac disease, with a dissociation constant (K_{d}) at least 10 times less than the copolymer's K_{d} for binding HLA-DR molecules and/or other DQ isotypes. Such HLA-DQ molecules are the combined protein products of specific HLA-DQB1 and DQA1 alleles known as DQB1*0201, DQB1*0302, DQB1*0304, DQB1*0401, DQB1*0501, DQB1*0502; and DQA1*0301, DQA1*0302, DQA1*0303, DQA1*0501. These alleles may be encoded on the same haplotypes (“cis” alleles) such as DQB1*0201-DQA1*0501-DRB1*0301 and DQB1*0302-DQA1*0301-DRB1*0401. The resulting HLA molecule comprising polypeptide products of “cis” alleles are referred to as “cis dimer.” Alternatively, the alleles may be encoded on different haplotypes (“trans” alleles). The HLA molecule comprising polypeptide products of “trans” alleles are referred to as “trans” dimer. An example of “trans” alleles is the combination of DQB1*0201 on DQB1*0201-DQA1*0501-DRB1*0301 and DQA1*0301 on DQB1*0301-DQA1*0301-DRB1*0404.

In certain embodiments, the DQ-directed RSPs used in the methods described herein are a mixture of randomized or partially randomized amino acid sequence containing amino acids from each of the following four groups: (1) hydrophobic, aliphatic amino acids (such as leucine, isoleucine, valine, methionine); (2) amino acids with acidic side chains (such as aspartic acid, glutamic acid); (3) amino acids with small hydrophilic side chains (such as serine, cysteine, threonine); and (4) amino acids with small aliphatic side chains (such as alanine, glycine); additionally, the copolymer contains proline residues. In one embodiment, the copolymer is derived using the amino acids Glutamine (E) and/or Aspartic acid (D), Leucine (L), Serine (S) and Alanine (A), and is referred to herein as an “ELSA” copolymer.

In certain other embodiments, the DQ-directed RSPs are a mixture of randomized or partially randomized amino acid sequence containing amino acids from each of the following four groups: (1) hydrophobic, aliphatic amino acids (such as leucine, isoleucine, valine, methionine); (2) bulky hydrophobic amino acids (such as tyrosine, phenylalanine, leucine, methionine); (2) amino acids with acidic side chains (such as aspartic acid, glutamic acid); (3) amino acids with small hydrophilic side chains (such as serine, cysteine, threonine); and (4) amino acids with small aliphatic side chains (such as alanine, glycine); additionally, the copolymer contains proline residues. An exemplary copolymer is derived using the amino acid residues Glutamine (E) and/or Aspartic acid (D), Leucine (L), Tyrosine (Y) and Val (V), and is referred to herein as an “DLYV” copolymer.

In particular embodiments, the RSP compositions useful for the present invention bind to one or more DQ isotypes with an average K_{d} of 1 μM or less, and more preferably an average K_{d} less than 100 nM, 10 nM or even 1 nM. Another way to identify preferred copolymers is based on the measure of a copolymer to displace another in competitive binding assays, such as described in Sidney et al., 2002, J. Immunol. 169:5098, which is expressed as an IC₅₀ value. Preferred RSPs of the present invention have IC₅₀'s less than 1 μM, more preferably less than 500 nM, and even more less than 100 nM.

In certain embodiments, particular RSPs of the present invention comprise amino acid residues K, E, A, S, V, and P. More preferably, the ratio of K:E:A:S:V is 0.3:0.7:9:0.5:0.5:0.3. Preferably, the RSPs are about 10 to 100 amino acid residues long, more preferably 20 to 80 amino acid residues long, even more preferably 40 to 60 amino acid residues long, and most preferably about 50 amino acid residues long. When synthesized, a typical preparation of RSPs is a mixture of peptides of various lengths, the majority of which are of the desired length but containing shorter or longer peptides inevitably created by the currently available synthetic processes.

Additional RSP for use in the present invention, and methods of synthesizing them, may be found in the literature, such as in Shukaliak Quandt, J. et al. (2004) Mol. Immunol. 40(14-15):1075-87; Montaudo, M S (2004) J. Am. Soc. Mass Spectrom. 15(3):374-84; Takeda, N. et al. (2004) J. Control Release 95(2): 343-55; Pollino, J M et al. (2004) J. Am. Chem. Soc. 126(2):563-7; Fridkis-Hareli, M et al. (2002) J. Clin Invest. 109(12):1635-43; Williams, D M et al. (2000) J. Biol. Chem. 275(49): 38127-30; Tselios, T. et al. (2000) Bioorg. Med. Chem. 8(8): 1903-9; and Cady, C T et al. (2000) J. Immunol. 165(4): 1790-8.

In certain embodiments, the RSPs useful for the instant invention are formulated for use as a medicament so as to have a polydispersity less than 25,000, and more preferably less than 10000, 5000, 1000, 500, 100, 50, or even less than 10.

Synthesis of RSPs

The RSPs used in the present invention can be made by any procedure available to one of skill in the art, and as previously disclosed. For example, the peptide synthesis process disclosed in U.S. Pat. No. 3,849,550, can be used wherein the N-carboxyanhydrides of tyrosine, alanine, γ-benzyl glutamate and N-ε-trifluoroacetyl-lysine are polymerized at ambient temperatures in anhydrous dioxane with diethylamine as an initiator. Briefly, any labile and/or reactive side chains are protected and Fmoc and/or t-Boc modified amino acids are used to react to couple them to elongate peptide chains. One of skill in the art readily understands that the process can be adjusted to make peptides and polypeptides containing the desired amino acids, that is, three of the four amino acids in Cop-1, for example, by selectively eliminating the reactions that relate to any one of glutamic acid, alanine, tyrosine, or lysine. For purposes of this application, the terms “ambient temperature” and “room temperature” mean a temperature ranging from about 20 to about 26° C. A preferred synthesis method of the RSPs of the present invention is by solid phase synthesis.

An example of amino acid input ratios in a representative example of YFAK synthesis with progressively higher alanine contents is as follows:

| Positions | Y | F | A | K |
|---|---|---|---|---|
| 0-10 | 3.7 | 5.5 | 64.4 | 26.4 |
| 11-20 | 4.3 | 5.1 | 71.4 | 19.2 |
| 21-30 | 4.0 | 4.7 | 71.5 | 19.8 |
| 31-40 | 3.6 | 4.7 | 74.3 | 17.4 |
| 41-52 | 3.0 | 4.1 | 76.0 | 16.8 |

An example of amino acid input ratios in a representative example of YEAK synthesis with progressively higher alanine contents is as follows:

| Positions | Y | E | A | K |
|---|---|---|---|---|
| 0-10 | 3.7 | 9.1 | 21.4 | 22.0 |
| 11-20 | 4.3 | 8.5 | 23.8 | 16.0 |
| 21-30 | 4.0 | 8.0 | 23.9 | 16.5 |
| 31-40 | 3.6 | 7.8 | 24.8 | 14.5 |
| 41-52 | 3.0 | 6.8 | 25.3 | 14.0 |

More concretely, an exemplary synthesis of YFAK starts with preparing Fmoc-Ala-Wang, Fmoc-Lys (Boc)-Wang, Fmoc-Tyr (tBu)-Wang, Fmoc-Phe-Wang, and the Fmoc group is cleaved with 20% Piperidine/N-methylpyrrolidone (“NMP”). A mixture of diisopropylethylamine (“DIPEA”)/NMP, Fmoc-Ala-OH, Fmoc-Lys (Boc)-OH, Fmoc-Tyr (tBu)-OH, Fmoc-Phe-OH, O-(Benzotriazol-1-yl)-N,N,N′,N′-tetramethyluronium tetrafluoroborate (“TBTU”)/NMP are used for the coupling/elongation step, wherein the amino acids are present in the mixture in a prescribed ratio. In a particular composition useful in embodiments of the instant invention, the input molar ratio of Y:F:A:K is 1:1:10:6 to start the peptide synthesis. After the coupling reaction is done, any amino acids that were not incorporated into the peptide chain and residual reagents are washed away and a new batch of the mixture is added for the next cycle of peptide elongation reaction. The elongation process is carried out for a designated number of cycles of reaction and washing, each cycle using a mixture of amino acids in particular ratios. After a desired number of cycles, any further elongation is stopped by acetylating the N-terminus with acetic anhydride, the reactants are filtered, and the peptides are cleaved from the resin support using TFA (trifluoroacetic acid), at the same time removing protective groups of the side chains. The peptides are washed, precipitated by IPE (isopropyl ether), purified and salt exchanged (from TFA to acetic acid salt) by ion exchange column with the mobile phase consisting of ethanol, Ac-52-OH×TFA.

In one embodiment, the peptides comprising complex peptide mixtures useful for the practice of the present invention are composed of naturally-occurring amino acids. In other embodiments, the copolymers are comprised of naturally occurring and synthetic derivatives, for example, selenocysteine. Amino acids further include amino acid analogs and D-amino acids. An amino acid “analog” is a chemically related form of the amino acid having a different configuration, for example, an isomer, or a D-configuration rather than an L-configuration, or an organic molecule with the approximate size and shape of the amino acid, or an amino acid with modification to the atoms that are involved in the peptide bond, so as to be protease resistant when polymerized in a polypeptide. A preferred composition consists of L-amino acids.

In certain embodiments, the complex peptide mixtures useful for the present invention include such linear copolymers that are further modified by substituting or appending different chemical moieties. In one embodiment, such modification is at a residue location and in an amount sufficient to inhibit proteolytic degradation of the copolymer in a subject. For example, the amino acid modification may be the presence in the sequence of at least one proline residue; the residue is present in at least one of carboxy- and amino termini; further, the proline can be present within four residues of at least one of the carboxy- and amino-termini.

In certain embodiments, the peptides comprising complex peptide mixture is a peptidomimetic. Peptidomimetics are compounds based on, or derived from, peptides and proteins. The copolymer peptidomimetics of the present invention typically can be obtained by structural modification of one or more native amino acid residues, e.g., using unnatural amino acids, conformational restraints, isosteric replacement, and the like. The subject peptidomimetics constitute the continuum of structural space between peptides and non-peptide synthetic structures.

Such peptidomimetics can have such attributes as being non-hydrolyzable (e.g., increased stability against proteases or other physiological conditions which degrade the corresponding peptide copolymers), increased specificity and/or potency. For illustrative purposes, peptide analogs of the present invention can be generated using, for example, benzodiazepines (e.g., see Freidinger et al. in “Peptides: Chemistry and Biology,” G. R. Marshall ed., ESCOM Publisher: Leiden, Netherlands, 1988), substituted gamma lactam rings (Garvey et al. in “Peptides: Chemistry and Biology,” G. R. Marshall ed., ESCOM Publisher: Leiden, Netherlands, 1988, p123), C-7 mimics (Huffman et al. in “Peptides: Chemistry and Biology,” G. R. Marshall ed., ESCOM Publisher: Leiden, Netherlands, 1988, p. 105), keto-methylene pseudopeptides (Ewenson et al. (1986) J. Med. Chem. 29:295; and Ewenson et al. in “Peptides: Structure and Function (Proceedings of the 9th American Peptide Symposium),” Pierce Chemical Co. Rockland, III., 1985), β-turn dipeptide cores (Nagai et al. (1985) Tetrahedron Lett. 26:647; and Sato I. (1986) J. Chem. Soc. Perkin Trans. 1:1231), β-aminoalcohols (Gordon et al. (1985) Biochem. Biophys. Res. Commun. 126:419; and Dann et al. (1986) Biochem. Biophys. Res. Commun. 134:71), diaminoketones (Natarajan et al. (1984) Biochem. Biophys. Res. Commun. 124:141), and methyleneamino-modified (Roark et al. in “Peptides: Chemistry and Biology,” G. R. Marshall ed., ESCOM Publisher: Leiden, Netherlands, 1988, p 134). Also, see generally, Session III: Analytic and synthetic methods, in “Peptides: Chemistry and Biology,” G. R. Marshall ed., ESCOM Publisher: Leiden, Netherlands, 1988).

### Improvements in Copolymer Therapy

As described in the Background section herein, therapy using RSP can be more effective and be developed rationally using the methods of the instant invention. The methods of the instant invention provide a means to correlate direct copolymer measurements with a given functionality in a given subject. Such correlation would allow more appropriate dosing, and therefore, improvements in copolymer therapy. This aspect is particularly important in treatment using complex peptide mixtures, because improper dosage can result in undesired immune responses contrary to the treatment modality.

Complex peptide mixtures useful practicing this invention are described in the section above entitled COMPLEX PEPTIDE MIXTURE, including the process to manufacture such complex peptide mixtures. Such complex peptide mixtures include RSPs consisting of Y, E, A, and K in certain advantageous output molar ratios such as Y:E:A:K=1.0:2.0:6.0:5.0, and higher molar alanine content; or Y, F, A, and K in output molar ratios such as Y:F:A:K=1.0:1.2:Xa:6.0 wherein 20.0<Xa<30.0.

An aspect of the instant invention is to provide methods of administering therapeutic and safe amounts of complex peptide mixtures to a mammalian subject, such amount based on the bioavailable portion of the dosed amount as determined by the method of quantitative detection described herein.

In certain embodiments, a complex peptide mixture of interest is administered to a subject in doses starting from about 0.001 mg/kg to about 80 mg/kg in a dose in a bolus administration. In addition, a control subject is administered the same composition only lacking complex peptide mixture. Administration is carried out at a frequency of about every 1, 2, 3, 4, 6, 12, 18, 24, 36, 48, or 72 hours, or alternatively daily, every other day, every third day, weekly, biweekly, monthly, every 2, 3, 4, 5, or 6 months. Preferably, a dose in adult human is from about 0.05 mg to 20 mg per dose, and more preferably, from about 1 mg to 15 mg per dose. Particularly, a dose in adult human is 1, 2, 3, 4, 5, 7, 10, 11, 12, 13, 14, or 15 mg per dose. Alternatively, administration is continuous over time through a sustained release formulation or device, wherein the total dosage is less than that achieved by daily bolus administration, i.e., less than about 0.001 mg/kg to up to about 80 mg/kg. Routes of administration include but are not limited to intravenous, subcutaneous, intramuscular, intradermal, intraperitoneal or intradermal or oral administration. Other examples of forms and routes of administration of an RSP composition are described, among others, in a PCT application publication WO 2007/059342.

In case of sustained release formulation or device, in preferred embodiments, the sustained release formulation administers the copolymer over a period of at least 2, 3, 4, 5, 6, 7, 8, 9, 10, 11, 12, 13 or 14 days; 3, 4, or 6 weeks; 2, 3, 4, 5, or 6 months. In another embodiment, the total dosage delivered daily by the sustained release formulation is less than 90%, 80%, 70%, 60%, 50%, 40%, 30%, 20%, 10% or 5% of a daily dosage known to be effective in the treatment of the disease. In an specific embodiment, the sustained release formulation administers 25% or less, per day, of a dosage of a random copolymer which is known to be effective in treating the disease when administered daily. As an illustrative example, if Copolymer 1 (YEAK) is known to be effective in the treatment of relapsing-remitting multiple sclerosis when administered daily in dosages of 20 mg, such as by one daily subcutaneous injection of 20 mg, the invention provides sustained release formulations of Copolymer 1 which results in a daily administration of copolymer of less than 20 mg, and in particular less than about 10 mg, 9 mg, 8 mg, 7 mg, 6 mg, 5 mg, 4 mg, 3 mg, 2 mg or 1 mg of Copolymer 1.

In some embodiments, sustained release compositions include formulation in lipophilic depots (e.g., fatty acids, waxes, oils), in suspension in water-in-oil microparticles and/or emulsions. The oil may be any non-toxic hydrophobic material liquid at ambient temperature to about body temperature, such as edible vegetable oils including safflower oil, soybean oil, corn oil, and canola oil; or mineral oil. Chemically defined oil substance such as lauryl glycol may also be used. The emulsifier useful for this embodiment includes Span 20 (sorbitan monolaurate) and phosphatidylcholine. In some embodiments, a RSP is prepared as an aqueous solution and is prepared into an water-in-oil emulsion dispersed in 95 to 65% oil such as mineral oil, and 5 to 35% emulsifier such as Span 20. In another embodiment of the invention, the emulsion is formed with alum rather than with oil and emulsifier. These emulsions and microparticles reduce the speed of uptake of RSP, and achieves controlled antigen delivery. In other embodiments, sustained release administration is achieved by using a device such as implanted sustained-release capsule or a coated implantable medical device.

Tissue samples are taken from the subject and the amount of complex peptide mixture is determined as described in the above section entitled PEPTIDE DETECTION AND QUANTITATION. In a particular embodiment, the tissue sample is blood, or serum or plasma prepared from such blood. The bioavailable portion of the administered peptide mixture is determined by comparing the dosed amount and the amount detected by the method described herein.

In particular embodiments, the method further comprises determining changes of physiological markers between the control sample and test samples by comparing the two results to assess the pharmacodynamic effect of the administered complex peptide mixture. In certain embodiments, physiological markers include but are not limited to hormones, enzymes, serum proteins, cytokines, immunomodulators, or an effector or regulator of any of these functional proteins. More particularly, physiological markers for safety, i.e., to detect unfavorable changes to avoid adverse effect, include Tryptase, IL-2, IL-3, IL-4, IL-6, IL-10, IL-23, IL25, IL-17, IL-27, TNFa, IFNg, antibodies including IgA, IgE, IgG1, IgG2, IgG3, and IgG4 reactive against the complex peptide mixture, and total IgE serum level. These markers are generally associated with inflammatory-type immune response activation, expressed by “type I” monocytes. Markers for efficacy include CXCL13, BDNF, CD40, CD40L, IFNγ, IL-1α, IL-1β, IL-1ra, IL-4, IL-5, IL-6, IL-10, IL-12p40, IL-12p70, IL-13, IL-15, IL-16, CCL22, MMP-2, MMP-3, MMP-9, TIMP-1, TNFα, TNFβ, and TNF-RII, as well as CD4, CD8, CD14, CD11b, CD44, CD45RA, CD45RO, CD27, CD123, CD127, CCR5, CCR9, FoxP3, and CCR7. These markers are associated with regulatory-type immune response activation, expressed by “type II” monocytes. Changes in these markers can be determined using commercially available kits, well known to one skilled in the art. Examples of means of quantitative detection include immunological methods such as ELISA or Western blot, enzymological methods such as reactions using substrates the product of which can be detected by colorimetric, fluorimetric, luminescence, or radioisotopic measurement, biological methods such as assays measuring proliferation, differentiation, cell cycle arrest, cell death, or cell division (either mitotic or meiotic). A statistically significant change is meaningful, and any change greater than 1.5 times, a 2 times, a 3 times, a 4 times, a 5 times or greater increase/decrease in either expression, activity, or serum concentration may be considered meaningful depending on the marker.

In one embodiment, said subject is a rodent. In particular embodiments, the subject is mouse. In other particular embodiments, the subject is rat. In another embodiment, said subject is human.

Alternatively or in addition to observing changes in the pharmacodynamic parameters, in other embodiments, behavioral changes or changes in symptoms of a disease or a condition, or other evidence of the effect of administration are observed.

In a more particular embodiment, the steps described immediately preceding hereto are carried out using experimental subjects. By choosing a parameter known to correlate between the experimental subjects and therapeutic subjects, an effect of a particular dose on the therapeutic subjects are better predicted. In certain embodiments, such experimental subjects are rodents. Rodent includes but is not limited to mouse, rat, rabbit, guinea pig, and hamster. In other embodiments, said mammal is a dog, a mini-pig or other micro swine, a ferret, a primate, a cat, a sheep, a goat, or a horse. In another particular method, said primate is a cynomoglus monkey, a rhesus monkey, or a human.

A further aspect of the instant invention is to provide methods to predict a therapeutically optimal amount of complex mixture to be delivered to a therapeutic subject (particularly a human subject) based on data obtained from experimental subjects. Such method comprises the steps of administering therapeutically optimal amounts of complex peptide mixtures to a non-human experimental mammalian subject, determining the bioavailable portion of the dosed amount using the method of quantitative detection described herein, determining functional read outs, and predicting a therapeutically optimal amount of complex peptide mixture to be delivered to the therapeutic subject based on the data obtained for the experimental mammalian subject and established correlation between the therapeutic and experimental subjects. The methods to determine the functional readout in response to administration of complex peptide mixture compositions are outlined in the paragraph immediately preceding this paragraph. In particular embodiments, the experimental subject is a rodent. In particular embodiments, the experimental subject is mouse. In other particular embodiments, the experimental subject is rat. The optimal amount of a complex peptide mixture to attain a desired outcome in a therapeutic subject is then predicted based on a known or experimentally determined correlation between one or more functional readouts of the experimental subject and that of the therapeutic subjects.

Yet another aspect of the instant invention is to provide an efficient and effective methods of treating a patient by administering a complex peptide mixture, such methods comprising the steps of: synthesizing peptides consisting of a complex peptide mixture by peptide synthesis, preparing a pharmaceutically acceptable formulation of said complex peptide mixture, administering said complex peptide mixture to a subject, obtaining a tissue sample from said subject, determining the amounts and/or concentrations of the complex peptide mixture in said tissue sample, determining a functional readout, correlating the said amounts of the complex peptide mixture to the functional readout, and optimizing the dosage of said complex peptide mixture to the subject by attaining the optimal functional readout. For the purposes of the instant invention, a functional readout can be the phenotype or function of the subject, the phenotype or function of cellular material derived from the subject, or the composition of fluids derived from the subject. In a particular embodiment, the detection step is repeated after certain time intervals to determine the time-course of bioavailability after administration. In certain embodiment, a half-life of the complex peptide mixture as a group is determined from such time course.

### Improvements in Method of Manufacturing Random Sequence Copolymers

Improvements can also be made using the method of the instant invention to monitor and control batch-to-batch variations that plague the current production of certain RSPs including Copolymer-1, and to test any bioequivalence of copolymers made by slightly varying manufacturing protocols. As such, another aspect of the invention is a method of improving the manufacturing process of a composition comprising a complex peptide mixture, such method comprising preparing a complex peptide mixture according to a protocol, further preparing a composition comprising said complex peptide mixture, determining the bioavailable amount of complex peptide mixture in said composition by detecting the level or degree of functional readout, comparing such readout against a standard, and adjusting the protocol or the step of preparing the composition to obtain a desired bioavailability.

### DEFINITIONS

The term “antibodies” means any immunoglobulin peptides, including but not limited to IgG, IgM, IgA, IgE, and IgD from any species or any fragments or any modified and/or engineered peptides derived from immunoglobulin, both single chain and multiple-chained, that (1) recognize a molecular structure comprising a target, (2) bind to the target by interacting with at least part of the molecular structure, and either (3) alter the physiological activity of the target or (4) alter the reaction of a subject that harbors the target towards the target. Antibodies may be chimeric, for example as in humanized antibodies, and antibodies may be engineered by site directed mutagenesis of the CDR region of a naturally occurring peptide. Antibodies include not only full length and peptides that comprise the hypervariable region of a native immunoglobulin such as Fab and Fab′ fragments, but also short synthetic or engineered peptides that comprise the binding regions of naturally occurring antibodies, whether the binding regions comprise contiguous or noncontiguous peptide sequences. In the latter case, the synthetic or engineered peptides would comprise the peptide sequences of originally noncontiguous amino acid stretch as one contiguous sequence.

The term “associated with” means “coexistent with” or “in correlation with.” The term does not necessarily indicate causal relationship, though such relationship may exist.

The term “binding” refers to a direct association between two molecules, due to, for example, covalent, electrostatic, hydrophobic, ionic and/or hydrogen-bond interactions under physiological conditions, and including interactions such as salt bridges and water bridges.

The term “MHC molecule” means any class II major histocompatibility complex glycoproteins. Human MHC proteins are sometimes referred to as “HLA.”

The term “immunomodulation” means the process of increasing or decreasing the immune system's ability to mount a response against a particular antigenic determinant through the T-cell receptor (“TCR”)'s recognition of complexes formed by major histocompatibility complex (“MHC”) and antigens.

The term “immunosuppression” means the depression of immune response and reactivity in recipients of organ or bone marrow allotransplants.

The term “MHC activity” refers to the ability of an MHC molecule to stimulate an immune response, e.g., by activating T cells. An inhibitor of MHC activity is capable of suppressing this activity, and thus inhibits the activation of T cells by MHC. In preferred embodiments, a subject inhibitor selectively inhibits activation by a particular class 11 MHC isotype or allotype. Such inhibitors may be capable of suppressing a particular undesirable MHC activity without interfering with all MHC activity in an organism, thereby selectively treating an unwanted immune response in an animal, such as a mammal, preferably a human, without compromising the animal's immune response in general.

The term “patient” refers to an animal, preferably a mammal, including humans as well as livestock and other veterinary subjects in need of a therapeutic treatment, prophylactic treatment, or diagnostic procedure related to a disease or an undesirable condition.

The terms “peptide”, “polypeptide” and “protein” are used interchangeably herein. These terms refer to unmodified amino acid chains, and also include minor modifications, such as phosphorylations, glycosylations and lipid modifications. The terms “peptide” and “peptidomimetic” are not mutually exclusive and include substantial overlap.

A “peptidomimetic” includes any modified form of an amino acid chain, such as a phosphorylation, capping, fatty acid modification and including unnatural backbone and/or side chain structures. A peptidomimetic comprises the structural continuum between an amino acid chain and a non-peptide small molecule. Peptidomimetics generally retain a recognizable peptide-like polymer unit structure. Thus, a peptidomimetic may retain the function of a peptide it is structurally related to, such as binding to an MHC protein forming a complex which activates autoreactive T cells in a patient suffering from an autoimmune disease.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g. modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Most of the amino acids used in the complex peptide mixture of the present invention may exist in particular geometric or stereoisomeric forms. In preferred embodiments, the amino acids used to form the complex peptide mixtures used in the present invention are (L)-isomers, although (D)-isomers may be included in the complex peptide mixtures such as at non-anchor positions or in the case of peptidomimetic versions of the complex peptide mixtures.

“Prevent”, as used herein, means to delay or preclude the onset of, for example, one or more symptoms, of a disorder or condition.

“Treat”, as used herein, means at least lessening the severity or ameliorating the effects of, for example, one or more symptoms, of a disorder or condition.

“Treatment regimen” as used herein, encompasses therapeutic, palliative and prophylactic modalities of administration of one or more compositions comprising one or more complex peptide mixture compositions. A particular treatment regimen may last for a period of time at a particular dosing pattern, which will vary depending upon the nature of the particular disease or disorder, its severity and the overall condition of the patient, and may extend from once daily, or more preferably once every 36 hours or 48 hours or longer, to once every month or several months.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003; PHARMACOLOGY A Pathophysiologic Approach Edited by Josehp T. DiPiro, Robert Talbert, Gary, Yee, Gary Matzke, Barbara Wells, and L. Michael Posey. 5th edition 2002 McGraw Hill; Pathologic Basis of Disease. Ramzi Cotran, Vinay Kumar, Tucker Collins. 6th Edition 1999. Saunders.

### EXAMPLES

### Example 1

### Direct competition Enzyme Linked Immunosorbent Assay

A direct competition ELISA was carried out for the detection and quantitation of PI-2301, an RSP composition. PI-2301 was immobilized on a 96-well microtiter plate by applying a 4 ug/ml solution in a coating buffer (pH 9.5) to the wells and incubating overnight at 4° C. then blocked for 2 hours and washed to remove unbound proteins. Mouse serum containing known or unknown concentrations of PI-2301 were added to the washed plates along with Protein A purified biotinylated anti-2301 and incubated for 2 hours on a plate shaker. Unbound material was washed away and diluted streptavidin-horseradish peroxidase (HRP) conjugate was added to the wells. After washing away any unbound streptavidin conjugate, substrate for HRP catalyzed hydrolysis was added to the wells and incubated, yielding a blue color that turns yellow when stop solution is added. The optical density of the color was measured at 450 nm using a microtiter plate reader (for example, Bio-Tek ELx405) and a standard curve was generated. The intensity of the color measured is proportionate to the amount of biotinylated anti-PI2301 antibody bound by the immobilized PI-2301. The PI-2301 in the serum sample competes for the anti-2301 antibody, and therefore the more PI-2301 is in the serum, the less intense the color would be.

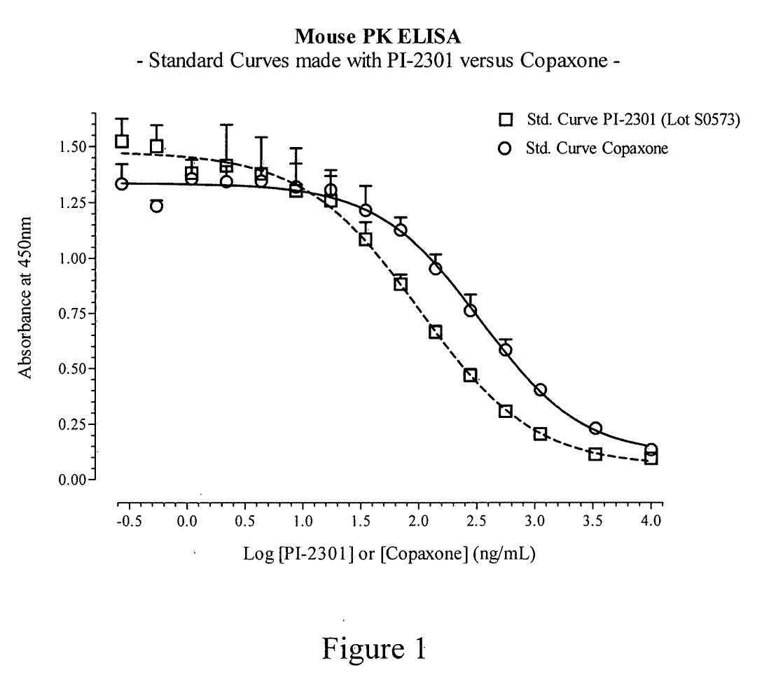

The resulting standard curve is shown in FIG. 1. The antibody for PI-2301 detects Cop-1 efficiently as well, allowing for the comparison of the two RSP compositions.

### Example 2

### Intra- and Inter-Assay Precision of the ELISA Detection of PI-2301 and Cop-1

The direct competition ELISA described in Example 1 was used to confirm the quantitative detection PI-2301 and COP-1 in mice serum by directly dosing normal, untreated mice serum with known amounts of the RSP compositions. To further qualify the method, various lots of pooled male CD1 mouse serum were spiked with PI-2301 and tested in this ELISA for precision, accuracy, specificity, linearity, limits of quantitation, and limits of detection. A minimum of three assays were run to generate the qualification data.

Data pertaining to the intra-assay precision for PI-2301 mouse serum spikes are shown in Table 1. For each of 3 days, the average, SD, and % RSD were obtained.

TABLE 1
Summary of intra-assay precision data in CD1 mouse serum
Within-day Variability-PI-2301 Spiked Mouse serum

| Results for Triplicate plates run | | | |
|---|---|---|---|
| on 3 different days (n = 3) | | | |
| | ng/mL | ng/mL- | ng/mL- |
| | Avg | SD | % RSD |
| Day 1 | | | |
| 1000 ng/mL | 1130.70 | 86.50 | 7.65 |
| 250 ng/mL | 279.10 | 19.14 | 6.86 |
| 50 ng/mL | 59.53 | 7.37 | 12.38 |
| Day 2 | | | |
| 1000 ng/mL | 926.43 | 73.14 | 7.89 |
| 250 ng/mL | 252.37 | 59.64 | 23.63 |
| 50 ng/mL | 45.91 | 11.26 | 24.53 |
| Day 3 | | | |
| 1000 ng/mL | 928.78 | 57.90 | 6.23 |
| 250 ng/mL | 270.27 | 22.10 | 8.18 |
| 50 ng/mL | 60.67 | 10.79 | 17.78 |

Target Criteria-% RSD ≦ 30%. All of the PI-2301 spiked serum samples met the target criteria for intra-assay precision.

Data pertaining to the inter-assay precision for PI-2301 mouse serum spikes is shown in Table 2. For 3 plates run on 3 days, the average, SD, and % RSD were obtained.

TABLE 2
Summary of inter-assay precision data in CD1 mouse serum
Between-day Variability-PI-2301 Spiked Mouse serum

| Results for Triplicate | | | |
|---|---|---|---|
| plates (n = 9 plates) | | | |
| | ng/mL- | ng/mL- | ng/mL- |
| Days 1-3 | Avg | SD | % RSD |
| 1000 ng/mL | 995.31 | 117.26 | 11.78 |
| 250 ng/mL | 267.25 | 13.62 | 5.10 |
| 50 ng/mL | 55.37 | 8.21 | 14.83 |

Target Criteria- % RSD ≦ 30%. All of the PI-2301 spiked serum samples met the target criteria for inter-assay precision.

Accuracy measures the closeness between the measured value and the theoretical value of the analyte in the sample. For this report, accuracy is measured between the calculated values of the spiked serum sample from the standard curve compared to the expected values as determined by calculations from the PI-2301 working stock. Within-day (intra-) and between-day (inter-) accuracy was calculated as follows:

% Error=Mean value of calculated conc.−Expected conc.×100 Expected conc.

Data pertaining to the intra-assay accuracy for PI-2301 mouse serum spikes is shown in Table 3. For each of 3 days, the mean calculated concentration and % Error were obtained.

TABLE 3
Summary of intra-assay accuracy data in CD1 mouse serum
Within-day Accuracy-PI-2301 Spiked Mouse serum

| Results for Triplicate plates run | | | |
|---|---|---|---|
| on 3 different days (n = 3) | | | |
| | Mean | Mean | |
| | Calc. | Expected | |
| | Conc.- | Conc.- | |
| | ng/mL | ng/mL | % Error |
| Day 1 | | | |
| 1000 ng/mL | 1130.70 | 1000 | 13.07 |
| 250 ng/mL | 279.10 | 250 | 11.64 |
| 50 ng/mL | 59.53 | 50 | 19.06 |
| Day 2 | | | |
| 1000 ng/mL | 926.43 | 1000 | −7.36 |
| 250 ng/mL | 252.37 | 250 | 0.95 |
| 50 ng/mL | 45.91 | 50 | −8.18 |
| Day 3 | | | |
| 1000 ng/mL | 928.78 | 1000 | −7.12 |
| 250 ng/mL | 270.27 | 250 | 8.11 |
| 50 ng/mL | 60.67 | 50 | 21.35 |

Target Criteria- % Error ± 30%. All of the PI-2301 spiked serum samples met the target criteria for intra-assay accuracy.

Data pertaining to the inter-assay accuracy for PI-2301 mouse serum spikes is shown in Table 4. For 3 plates run on 3 days, the mean calculated concentration and % Error were obtained.

TABLE 4
Summary of intra-assay accuracy data in CD1 mouse serum
Between-day Accuracy-PI-2301 Spiked Mouse serum

| Results for Triplicate plates | | | |
|---|---|---|---|
| (n = 9 plates) | | | |
| | Mean | Mean | |
| | Calc. | Expected | |
| | Conc.- | Conc.- | |
| Days 1-3 | ng/mL | ng/mL | % Error |
| 1000 ng/mL | 995.31 | 1000 | −0.47 |
| 250 ng/mL | 267.25 | 250 | 6.90 |
| 50 ng/mL | 55.37 | 50 | 10.74 |

Target Criteria- % Error ± 30%. All of the PI-2301 spiked serum samples met the target criteria for inter-assay accuracy.

The intra- and inter-assay precision for mouse serum samples spiked with PI-2301 at 1000, 250, and 50 ng/mL was acceptable, with % RSD ranging from 5.1% to 24.53% (Tables 1 & 2). The intra- and inter-assay accuracy for these same samples was also acceptable, with % error ranging from −7.36 to 21.35 (Tables 3 & 4).

### Example 3

### Specificity of ELISA Assay to Detect PI-2301 and Cop-1

Specificity is the ability of a method to measure the material of interest to the exclusion of other similar compounds. In this method, specificity is a function of the anti-PI-2301 biotinylated antibody (bAb). If the bAb recognizes, and binds preferentially to, the compound in the serum the bAb will be washed away and the final A450 value will be low. If the bAb does not recognize the compound it will bind to the PI-2301 immobilized on the plate and the A450 value will be higher. Table 5 summarizes the A450 specificity data for 3 compounds spiked into mouse serum.

TABLE 5
Summary of intra-assay accuracy data in CD1 mouse serum

| Compounds spiked | | |
|---|---|---|
| into CD1 Mouse | Conc.- | Mean |
| serum | ng/mL | A450 |
| PI-2301 (Q1373) | 1000 | 0.308 |
| CO-23 | 1000 | 1.420 |
| Poly A, L | 1000 | 1.233 |

Table 5 shows the specificity of the bAb for PI-2301. The high A450 values for CO-23 and Poly (A, L) mean the bAb bound to the PI-2301 immobilized on the plate not the CO-23 (Y:F:A:K with the input ratio of 1:1:1:1) or Poly (A, L) in the serum. The low A450 value for PI-2301 (Q1373) means the bAb bound to the PI-2301 in the serum and not to the immobilized PI-2301.

The specificity of this method was evaluated by comparing mouse serum spiked with 1000 ng/mL PI-2301 (Q1373-YFAK at input ratio of 1:1.2:18:6) with mouse serum spiked with 1000 ng/mL of PI-2301 (CO-23-YFAK at 1:1:1:1) or Poly Alanine, Lysine (Sigma). Only the PI-2301(Q1373) competed successfully for the biotinylated anti-PI-2301 antibody (bAb) in the serum, leading to the low OD450 nm (0.308) signal typically seen at this concentration. CO-23 and Poly (A, L) did not compete for the bAb in the serum; as a result, most of the bAb bound to the PI-2301 immobilized on the plate, leading to a high OD450 nm (1.42 and 1.233, respectively).

### Example 4

### Linearity of ELISA Assay to Detect PI-2301 and Cop-1

Linearity of a method refers to the ability of a method to generate results that are proportional to the concentration of analyte in the sample. A standard curve was prepared in mouse serum with PI2301 concentrations from 10,000 ng/mL to 0.27 ng/mL. A serum blank was included with each standard curve. For this method, the linear portion of the curve is defined as being between the PI-2301 concentrations that have back-calculated values within 70-130% of the expected values. The linear range of the curve is that part of the linear curve (above the LOD) with % RSD≦30%. Table 6 summarizes the linearity data for 9 curves run over 3 days.

TABLE 6
Summary of Standard Curve Linearity Data in CD1 mouse serum

| | | Std. Curve | |
|---|---|---|---|
| | | (n = 9) | |
| | PI-2301- | % Recovery | Std. Curve |
| Standard | ng/mL | Mean | (n = 9) % RSD |
| 1 | 10000 | 71.103 | 23.098 |
| 2 | 3333.3 | 105.371 | 17.231 |
| 3 | 1111.1 | 102.175 | 11.405 |
| 4 | 555.6 | 108.766 | 12.968 |
| 5 | 277.8 | 102.283 | 8.180 |
| 6 | 138.9 | 102.212 | 5.594 |
| 7 | 69.44 | 97.326 | 5.466 |
| 8 | 34.72 | 93.166 | 8.223 |
| 9 | 17.36 | 118.455 | 23.324 |
| 10 | 8.68 | 108.332 | 36.677 |
| 11 | 4.34 | 121.077 | 67.832 |
| 12 | 2.17 | 119.430 | 62.721 |
| 13 | 1.09 | 839.166 | 63.440 |
| 14 | 0.54 | 156.529 | 83.768 |
| 15 | 0.27 | 837.675 | 77.621 |
| 16 | 0.00 | NA | NA |

Table 6 shows the results of 9 standard curve runs. There was acceptable recovery (70-130%) of PI-2301 from 10000 to 2.17 ng/mL with acceptable (≦30%) % RSD between 10000 and 17.36 ng/mL. Based on this data the linear portion of the curve is between 10000 ng/mL and 2.17 ng/mL and the linear range is between 3333 ng/mL and 17.36 ng/mL.

The linearity of the method was evaluated over a concentration range of 10000 to 0.27 ng/mL PI-2301 in mouse serum (Table 6). This method is linear between 10000 and 2.17 ng/mL with acceptable % Recovery of PI-2301 of 70-130%. The linear range of the method is between 3333 and 17.36 ng/mL with % RSD≦30%.

### Example 5

### Limits of Quantitation and Limits of Detection

The Limits of Quantitation (LOQ) are the highest (ULOQ) and lowest (LLOQ) concentrations of analyte in a sample that can be measured with an acceptable level precision and accuracy. For this method, recovery within 70-130% of the expected values with precision (% RSD)<30% and accuracy (% error)±30% (±40% for LLOQ) will be acceptable. Spiked samples were prepared in mouse serum with PI-2301 concentrations between 1000 ng/mL and 10 ng/mL. Table 7 summarizes the LOQ data for 9 runs over 3 days.

TABLE 7
Summary of LOQ data for PI-2301 in mouse serum

| Spiked | PI-2301- | Precision | Accuracy |
|---|---|---|---|
| Sample | ng/mL | % RSD (n = 9) | % Error |
| 1 | 1000 | 13.717 | −0.47 |
| 2 | 500 | 13.476 | 11.88 |
| 3 | 250 | 8.386 | 6.90 |
| 4 | 200 | 12.526 | −11.46 |
| 5 | 100 | 15.037 | 2.56 |
| 6 | 50 | 17.070 | 10.74 |
| 7 | 25 | 36.505 | −28.21 |
| 8 | 20 | 27.158 | −1.07 |
| 9 | 10 | 44.942 | 19.10 |

Table 7 shows the results of 9 runs of PI-2301 spiked mouse serum samples evaluated for the determination of the upper and lower limits of Quantitation. Precision and accuracy criteria were met for all PI-2301 concentrations between 1000 and 20 ng/mL.

For this competitive ELISA, the limit of detection (LOD) is the highest concentration of PI-2301 that gives an optical density (OD) which is significantly different from the background or, in this case, the non-specific binding (NSB). For this qualification LOD is defined as NSB+(3×SD) where the NSB are samples of PI-2301 spikes assayed without biotinylated antibody. Table 8 summarizes the LOD data for 3 runs.

TABLE 8
Summary of LOD data from 3 runs of PI-2301 spiked mouse
serum without anti-PI-2301 biotinylated antibody

| | | | | | 2301 | 2301 |
|---|---|---|---|---|---|---|
| | | 2301 | 2301 | 2301 | Spikes-w/o | Spikes-w/o |
| Spiked | PI-2301- | Spikes-w/o | Spikes-w/o | Spikes-w/o | bAb pI 1-3- | bAb pI 1-3- |
| Sample | ng/mL | bAb pI 1- A450 | bAb pI 2- A450 | bAb pI 3- A450 | Mean A450 | SD A450 |
| 1 | 1000 | 0.07 | 0.10 | 0.09 | 0.086 | 0.016 |
| 2 | 500 | 0.08 | 0.10 | 0.09 | 0.090 | 0.013 |
| 3 | 250 | 0.07 | 0.09 | 0.09 | 0.084 | 0.009 |
| 4 | 200 | 0.08 | 0.07 | 0.08 | 0.075 | 0.006 |
| 5 | 100 | 0.07 | 0.08 | 0.09 | 0.080 | 0.011 |
| 6 | 50 | 0.07 | 0.22 | 0.10 | 0.129 | 0.078 |
| 7 | 25 | 0.07 | 0.07 | 0.08 | 0.072 | 0.007 |
| 8 | 20 | 0.08 | 0.09 | 0.09 | 0.085 | 0.010 |
| 9 | 10 | 0.08 | 0.08 | 0.11 | 0.087 | 0.017 |

| Mean NSB for | Mean SD for | LOD for PI-2301 ELISA- |
|---|---|---|
| all PI-2301 spiked | all PI-2301 spiked | (NSB + (3 × SD))- |
| conc.-OD450 nm | conc.-OD450 nm | OD450 nm |
| 0.088 | 0.0186 | 0.144 |

Table 8 shows the results for 3 runs of mouse serum samples spiked with PI-2301 and incubated without biotinylated antibody in order to determine the non-specific binding and LOD of this method. The Mean NSB for all of the PI-2301 spiked serum samples was OD450 nm=0.088. The LOD for this method was calculated as OD450 nm=0.144.

The upper and lower limits of Quantitation (ULOQ and LLOQ) of the method were evaluated for accuracy (% error±30%) and precision (% RSD≦30%) over a concentration range of 1000 to 10 ng/mL PI-2301 in mouse serum. Accuracy was acceptable, with % Error ranging from −28.21 to 19.1 and precision was acceptable for all concentrations except 25 and 10 ng/mL (36.5 and 44.9% RSD respectively—Table 7). Based on this data the ULOQ and LLOQ for this method are 1000 ng/mL and 20 ng/mL, respectively. The limit of detection (LOD) for this method is OD₄₅₀ₙₘ=0.144 and was determined by assaying PI-2301 spiked mouse serum samples without bAb for levels of non-specific binding and using the mean (OD₄₅₀ₙₘ=0.088) and standard deviation (OD450 nm=0.0186) for the NSB to calculate the LOD (see Table 8)

### Example 6

### Detection of PI-2301 and Cop-1 in Mouse Serum after Administration of the RSP Composition to Mice

Having validated the detection method under controlled condition, the ELISA was used to detect PI-2301 RSP composition after in vivo administration of the RSP composition to mice. Mice were injected SC (subcutaneous) interscapularly (in the skin between the shoulder blades) at a dose volume of 100 uL/10 g with either 0.25, 2.5, 25, or 40 mg/kg PI-2301 formulated in Osmitrol 42 mg/mL, 0.25, 2.5, 25, or 40 mg/kg Cop-1 formulated in Mannitol 42 mg/mL, or Osmitrol Vehicle 42 mg/mL. PI-2301, 0.25 mg/kg is a SC dose that has not been shown to be efficacious in ameliorating the severity of disease in experimental autoimmune encephalomyelitis however, has been shown to be immunogenic. PI-2301, 2.5 mg/kg is a SC dose that has been shown to be immunogenic and efficacious in ameliorating the severity of disease in experimental autoimmune encephalomyelitis without inducing toxicity in mice. PI-2301, 25 mg/kg has been defined in a GLP study as the No Observed Adverse Effect Level (NOAEL) in CD-1 mice (report #550-161). PI-2301, 40 mg/kg is toxic, i.e., several mice die within a 14 day period when administered 40 mg/kg daily SC.

For plasma collection, 200 uL of blood was collected into purple top tubes (containing EDTA) with anti-coagulant, inverted several times, and then centrifuged at 4° C., 10,000 RPM, for 10 minutes. Plasma was collected, placed on dry ice, and then stored at −80° C. until further analysis.

For serum analysis, 400 uL of blood was collected into yellow top tubes (containing serum separator gel) which do not contain anti-coagulant. The blood sample was allowed to clot at room temperature for 15-30 minutes and then the tube was centrifuged at 4° C., 10,000 RPM, for 10 minutes in order to isolate serum. Serum was collected, aliquoted, and stored −80° C. for future testing.

Quantitation of each of PI-2301 and Cop-1 in serum of mice dosed SC with PI-2301 was carried out by a direct competition ELISA. Briefly, PI-2301 or Cop-1 was immobilized on a 96-well microtainer plate overnight at 40° C., then blocked for 2 hours and washed. Normal mouse serum containing known concentrations of PI-2301 for use as a standard curve, as well as serum isolated from mice primed with different doses of PI-2301 (which contain unknown concentrations of PI-2301) was added to the washed plates along with Protein A-purified biotinylated rabbit anti-2301 antibody or anti-Cop-1 antibody and incubated at room temperature for two hours on a plate shaker. Antibody prevented from binding to the plate by PI-2301 present in the serum was washed away and an optimal dilution of streptavidin-HRP conjugate was added to the wells. After washing away any unbound conjugate, substrate was added to the wells and incubated, yielding a blue color that turned yellow when stop solution was added. The optical density was measured at 450 nm and a standard curve was generated. The intensity of the color measured was proportional to the amount of biotinylated anti-2301 or anti-Cop-1 antibody bound to the immobilized PI-2301 or Cop-1, respectively. The serum concentration of PI-2301 or Cop-1 contained in experimental samples was determined by measuring the absorbance at 450 nm on the linear portion of the standard.

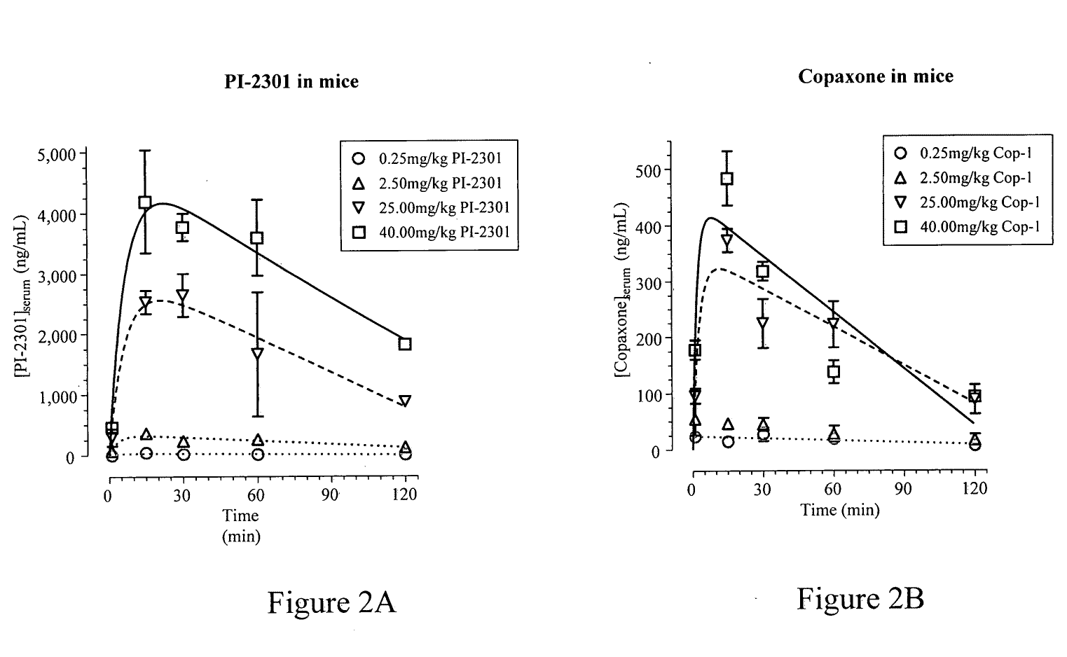

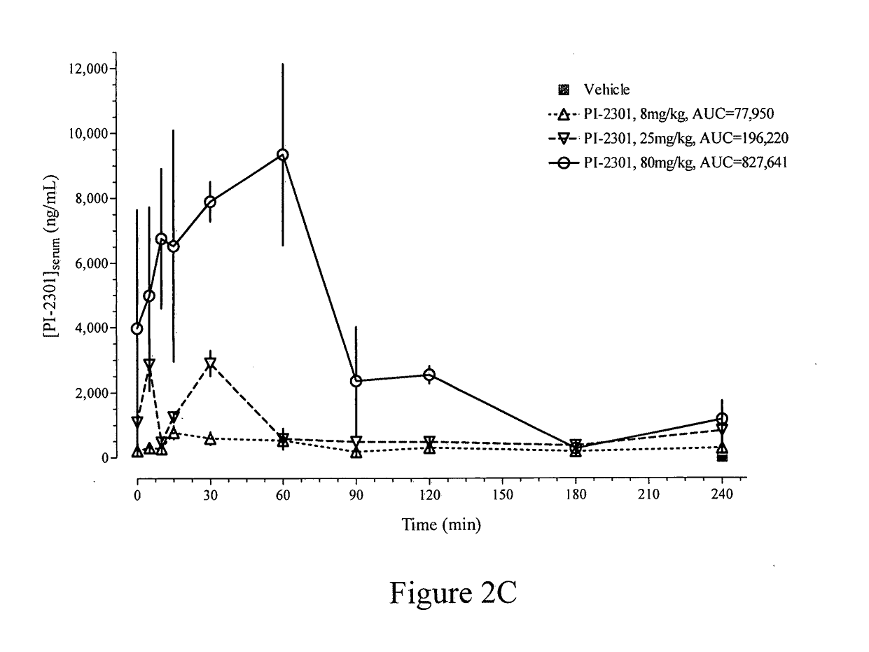

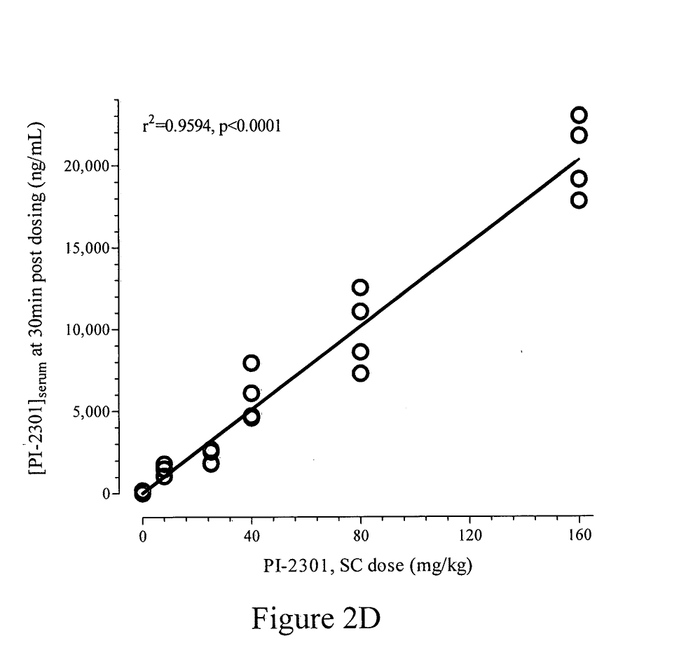

FIG. 2 Panel A shows the serum concentration of PI-2301, and Panel B shows that of Cop-1 after subcutaneous administration of the RSP to mice. The serum concentration peaks at about 15 minutes after the administration and decreases in a time dependent manner subsequently. The results indicate at, at the same dosage, PI-2301 concentration in the serum is approximately 10 times that of Cop-1. Further investigation with higher dosage of the peptide mixtures confirmed and extended this result for PI-2301 (Panel C). Panel D shows a linear correlation between an administered subcutaneous dose of PI-2301 and serum concentration of PI-2301 at 30 min in CD-1 mice.

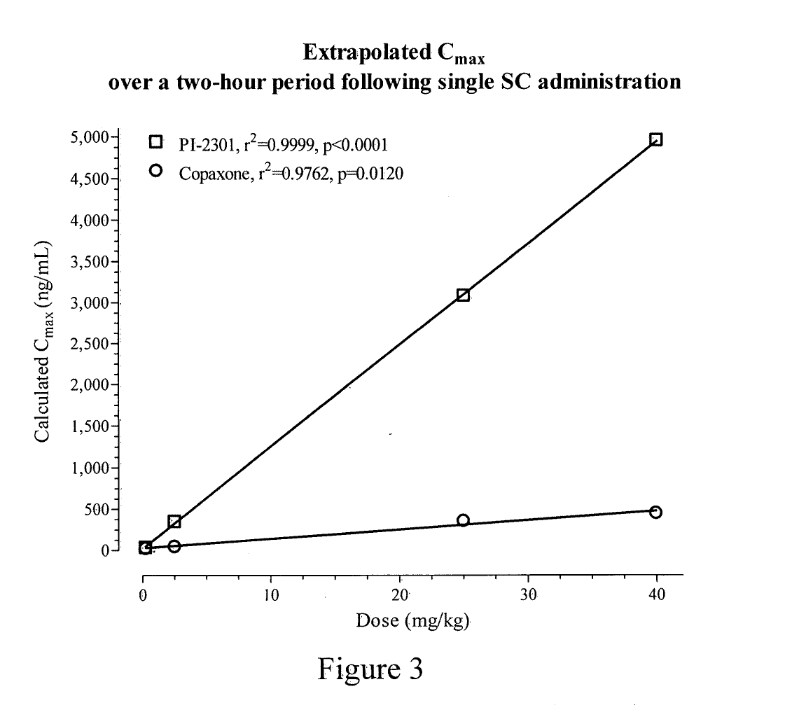

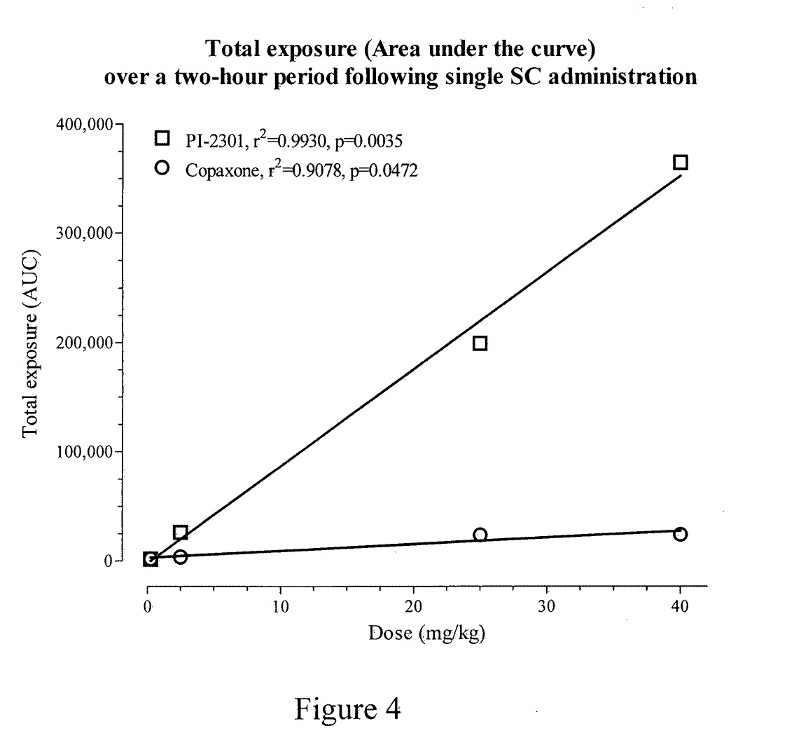

The data are also presented as the extrapolated maximal serum concentrations of the RSPs (FIG. 3) and the calculated total exposure of the mice to the RSP over time before the reagent is eliminated from the serum (FIG. 4). Both data indicate a much higher bioavailability of PI-2301 compared to Copaxone, even when the administered amount is the same, indicating the importance of measuring the actual serum concentration of the RSP after dosing.

### Example 7

### In Vitro Effects of PI-2301 and Cop-1 on Macrophages

To assess the usefulness of various potential functional readouts, the effect of PI-2301 and Cop-1 were investigated for several inflammatory and regulatory indicators in macrophages. Macrophage propagation and functional readouts were carried out by observing the effect of PI-2301, COP-1, or PLP139-151 on myeloid cells isolated from femurs of immunologically naive mice. Isolated cells were labeled with magnetic beads and T- and B-cells were depleted from the sample. Cells were cultured with PI-2301, Cop-1, or PLP139-151 in media containing 10% FBS, IL-3 10 ng/ml, and TNF-a 2.5 ng/ml, and supernatants were analyzed for cytokines and chemokines. TNFα, IL-6, CXCL1 and CXCL2 as inflammatory markers, and IL-12p70, CXCL13, and CCL22 as regulatory markers.

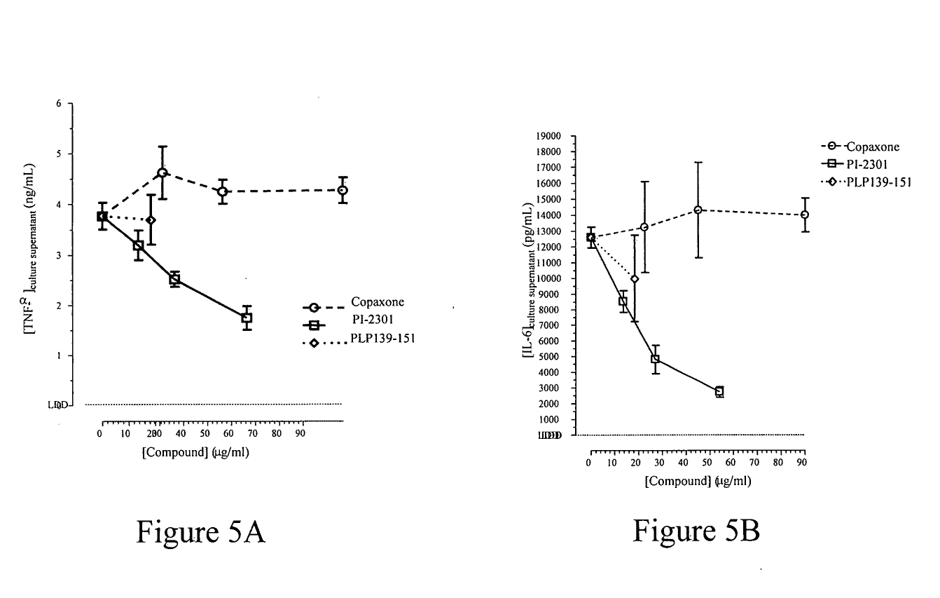

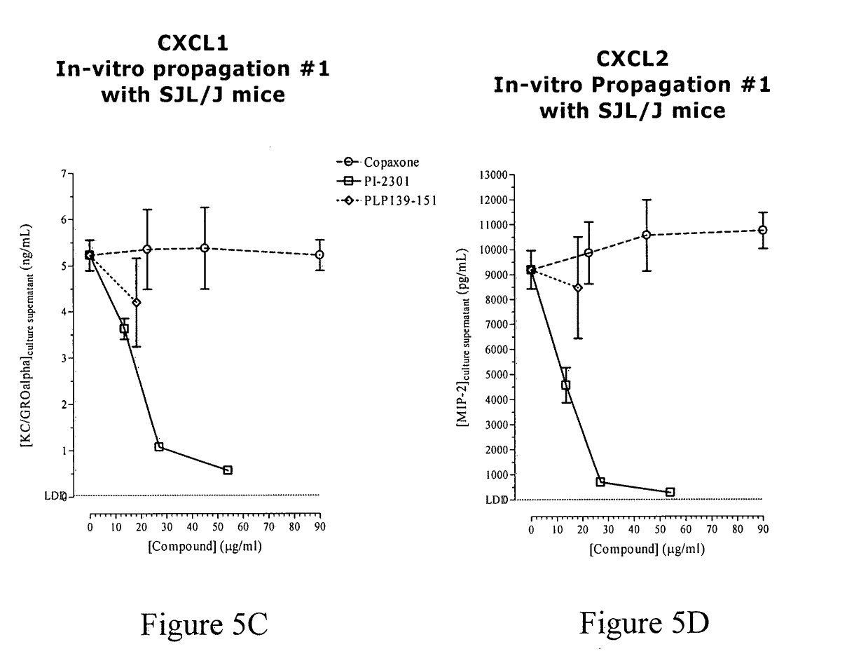

PI-2301 induced decrease in expression of TNFα (panel A), IL-6 (panel B), CXCL1 (panel C) and CXCL2 (panel D) as shown in FIG. 5A-D. In contrast, at the given dosage, Cop-1 had no significant effect on the expression of these factors.

### Example 8

### Pharmacodynamics of PI-2301 and Cop-1-Detection of CCL-22, CXCL10/IP-10, and CXCL13 from Blood Samples of Mice

The serum concentration of the complex peptide mixture is reflected in the physiological effects as well. After administration of a complex peptide mixture, blood was collected at 0, 15, 30, 60 and 120 minutes after administration, and processed as in Example 5 to obtain serum and plasma.

CCL-22 was detected using a purified monoclonal antibody specific for mouse pre-coated onto a 96-well plate. The serum, plasma, or cell culture supernatant samples was added to the wells, and any CCL22 present in the samples was bound by the immobilized anti-CCL22 antibody. On each plate a dose titration of a mouse CCL22 standard was also plated in order to generate a standard curve. After an overnight incubation unbound CCL22 was washed away and an anti-mouse CCL22:HRP conjugate was added to the wells for 2 hours. After washing away any unbound conjugate, a substrate was added to the wells and incubated for 30 minutes, yielding a blue color that turned yellow when stop solution was added. The optical density was measured at 450 nm and a standard curve was generated from the recombinant mouse CCL22 samples. CCL22 concentrations in the test serum, plasma, or cell culture supernatants was calculated from the standard curve.

CXCL10/IP-10 was detected using a commercial cytokine sandwich ELISA kit (Quantikine Mouse CXCL10/IP-10 Immunoassay, R&D Systems #MCX100). Anti-CXCL10 antibody was pre-coated onto a microplate. Cell supernatants and standards were incubated on the microplate and any unbound material was removed by washing. The bound CXCL10 was detected by a polyclonal antibody against mouse CXCL10, conjugated to horseradish-peroxidase. After a final wash to remove excess antibody, an enzymatic substrate composed of TMB (a chromogen) and hydrogen peroxide was added. The intensity product was directly proportional to the amount of CXCL10 present, and was measured spectrophotometrically with an ELISA plate reader.

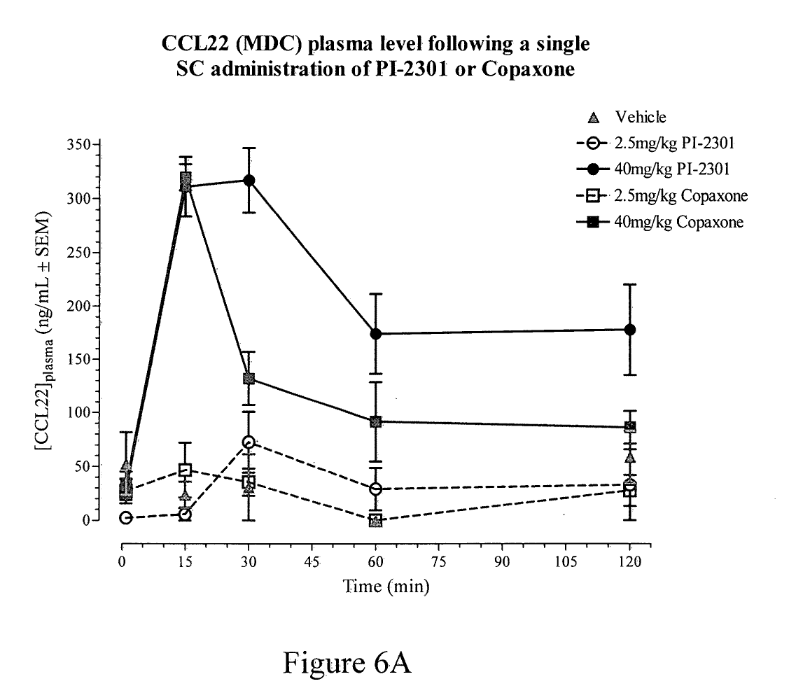

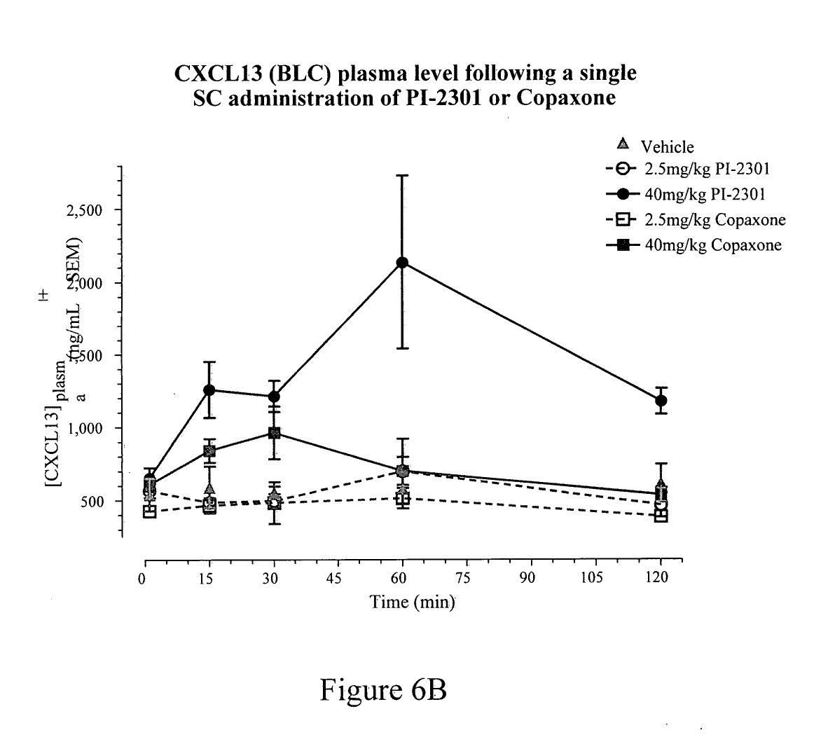

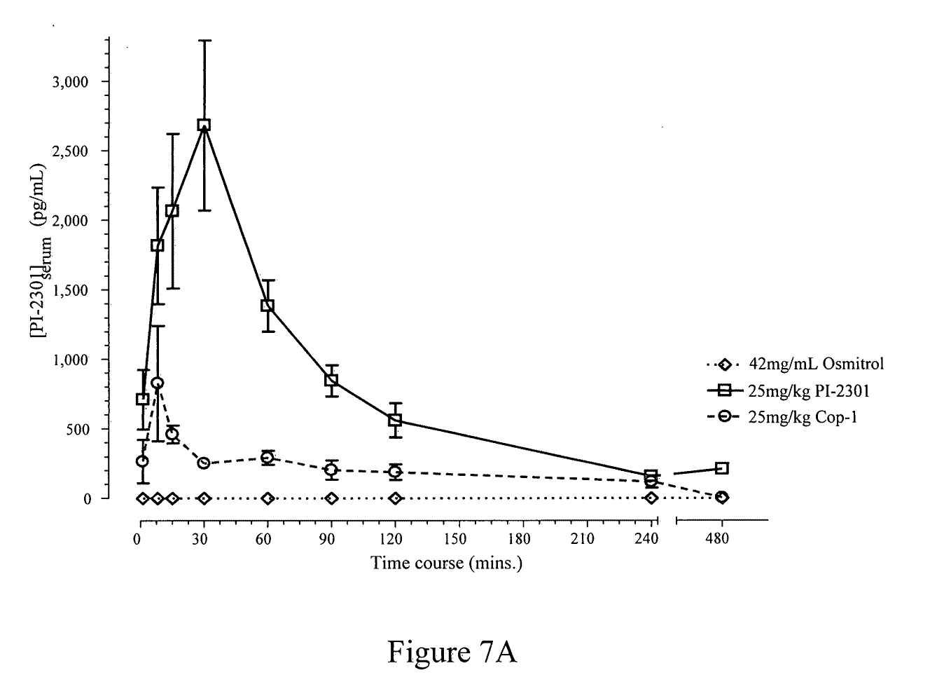

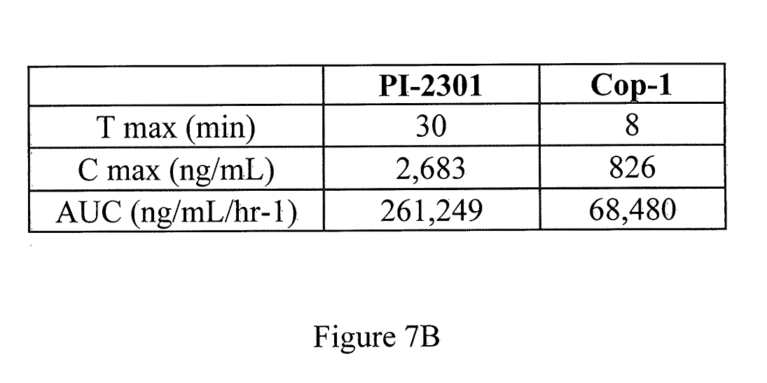

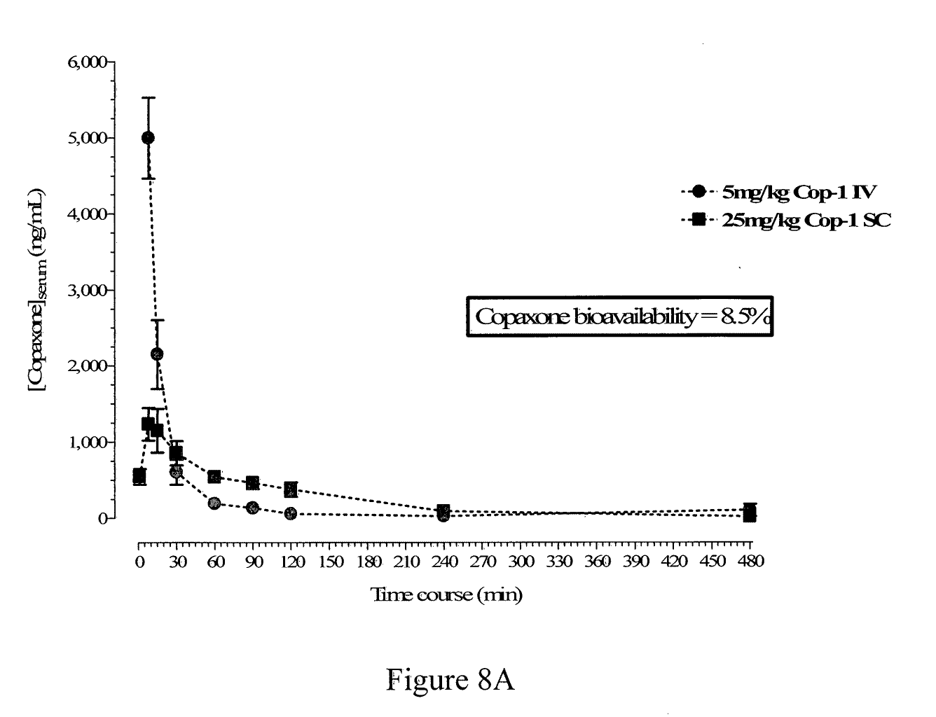

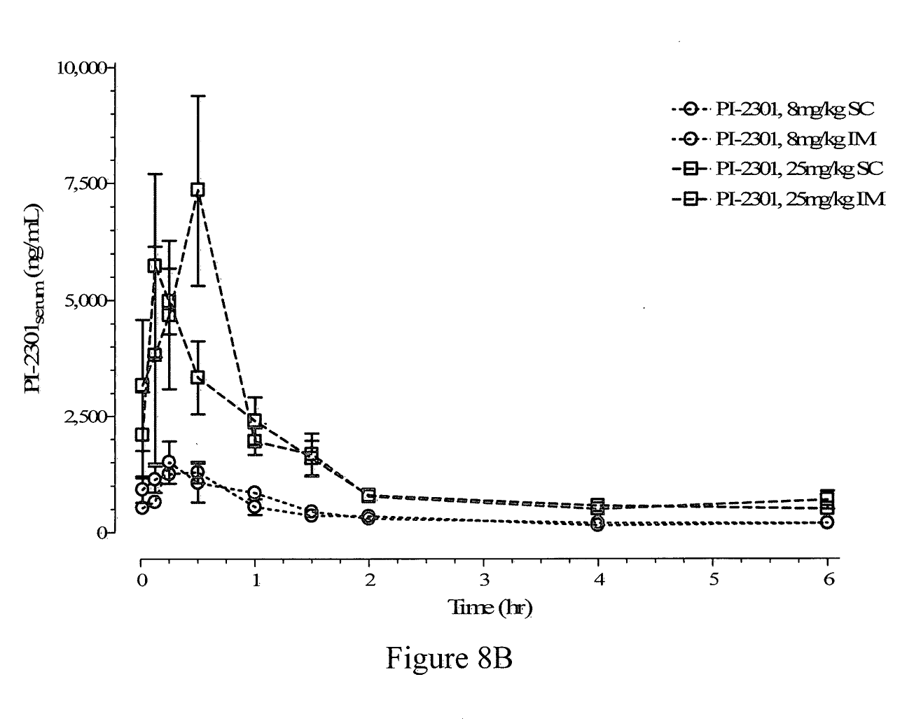

As shown in FIGS. 6A and 6B, indicators of the effect of the RSPs remain elevated even after time. Plasma levels of CCL22 (panel A) and CXCL13 (panel B) parallel compound serum exposure. In particular, CXCL13, a powerful B-cell chemoattractant, is detected at 3-fold higher level in PI-2301-treated mice compared to Copaxone-treated animals, 60 minutes after SC administration. The concentration of Copaxone and PI-2301 following a single subcutaneous administration of 25 or 80 mg/kg in mice is shown in FIG. 7A. The calculated parameters are shown in the table shown as FIG. 7B. Further, the bioavailability of Copaxone (FIG. 8A) following a single subcutaneous or intravenous administration and PI-2301 (FIG. 8B) a single subcutaneous and intramuscular administration in mice. About 8.5% of administered Copaxone is available in the plasma of mice. In contrast, about [ ]% of PI-2301 is available.

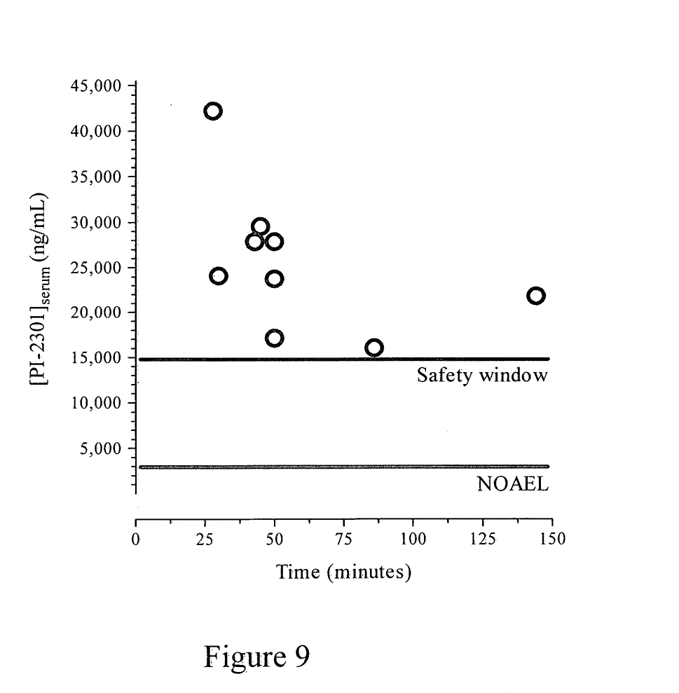

Further, during the experiments determining dose-dependent effects of PI-2301, adverse effects above certain serum concentration was observed. FIG. 9 shows that serum concentration above 15 μg/mL resulted in of death of mice indicated by circles.

### Example 9

### Detection of PI-2301 in Serum after Administration of the RSP Composition to Different Mammalian Species

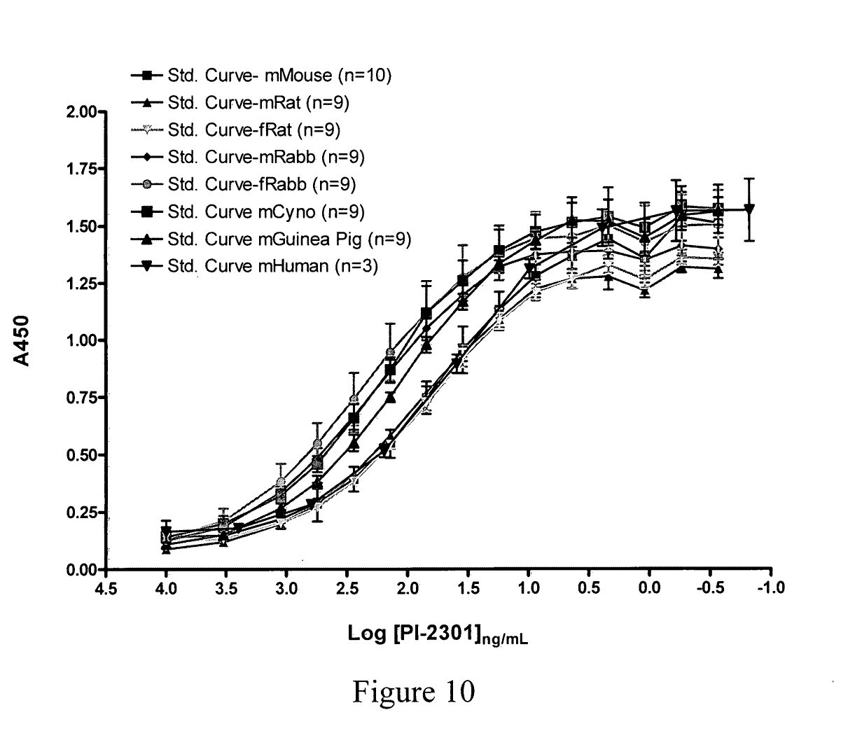

Because of different sensitivity to PI-2301 among different mammalian species, the bioavailability and serum concentration of PI-2301 was suspected to vary greatly for different mammalian species. First, the validity of the PI-2301 PK ELISA was confirmed for species other than mouse. FIG. 10 indicates the ELISA titration curve for different mammalian species. For all species tested (rat, rabbit, non-human primate (cynomolgus monkey), guinea pig, rabbit), the ELISA was valid with comparable assay linearity, precision, and accuracy.

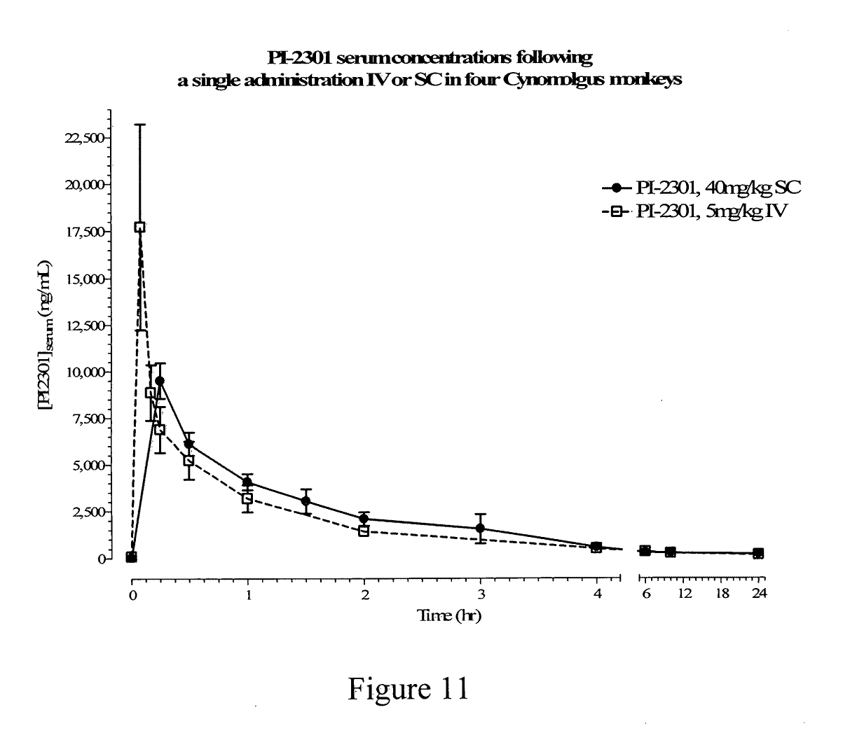

FIG. 11 shows the serum concentration of PI-2301 for cynomoglus monkeys. The bioavailability of PI-2301 is similar to mice at 13% after an intravenous administration (open square, 5 mg/kg) or a subcutaneous administration (closed circle, 40 mg/kg).

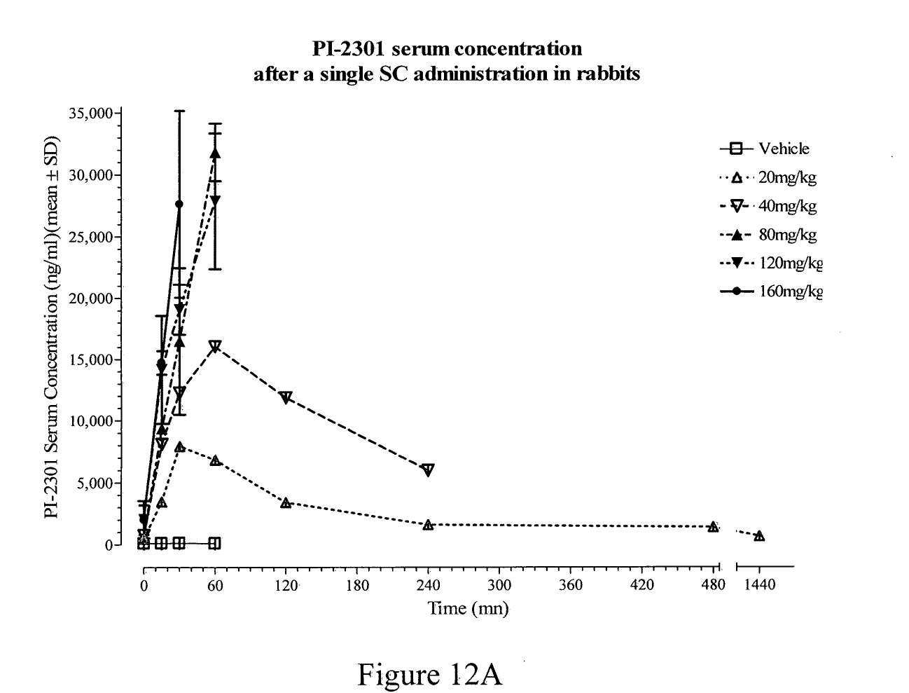

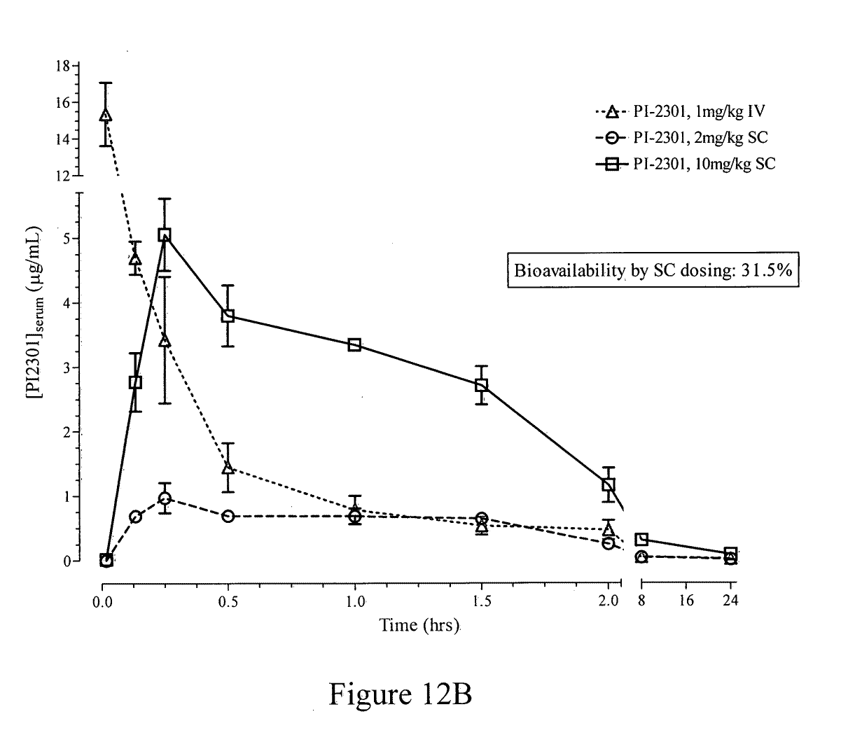

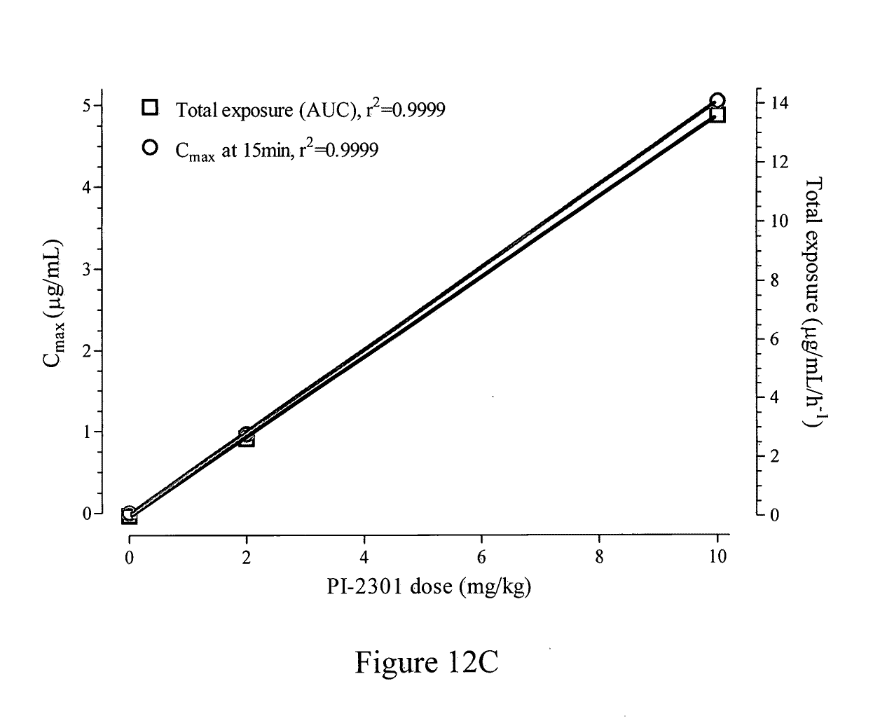

In contrast, FIG. 12 shows that the bioavailability is much higher in rabbits. Rabbits were dosed with a single subcutaneous administration of 20, 40, 80, 120, or 160 mg/kg of PI-2301. (FIG. 12A) Doses over 40 mg/kg caused death in less than 60 minutes after administration. FIG. 12B shows the serum concentration of PI-2301 after intravenous administration at 1 mg/kg and subcutaneous administration at 2 or 10 mg/kg. The bioavailability was calculated to be 31.5%, more than twice as high as that for mice and monkeys. FIG. 12C shows a clear linear correlation between dosed amount and total exposure shown by the area under the curve (AUC). The maximum exposure was at 15 minutes after administration.

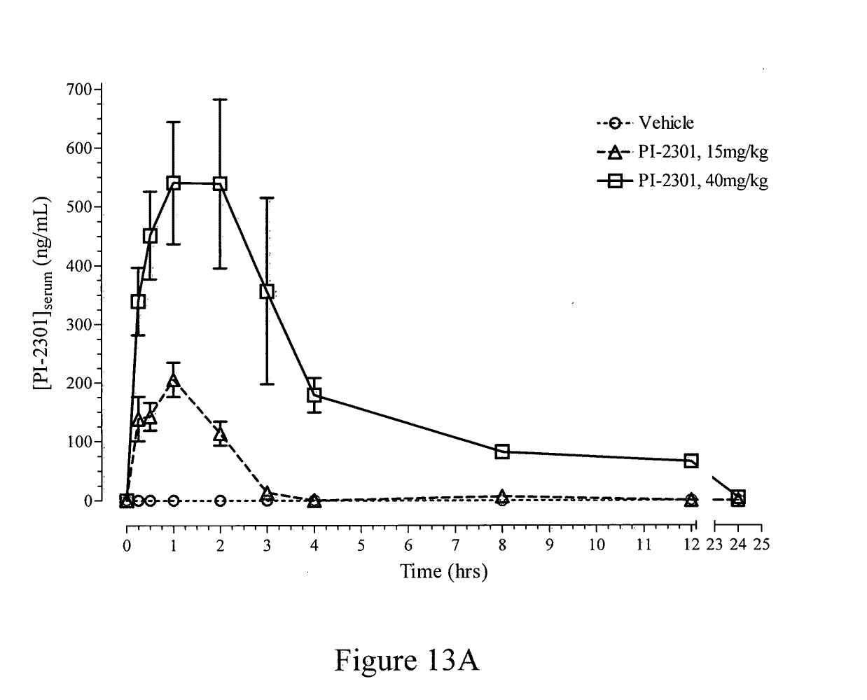

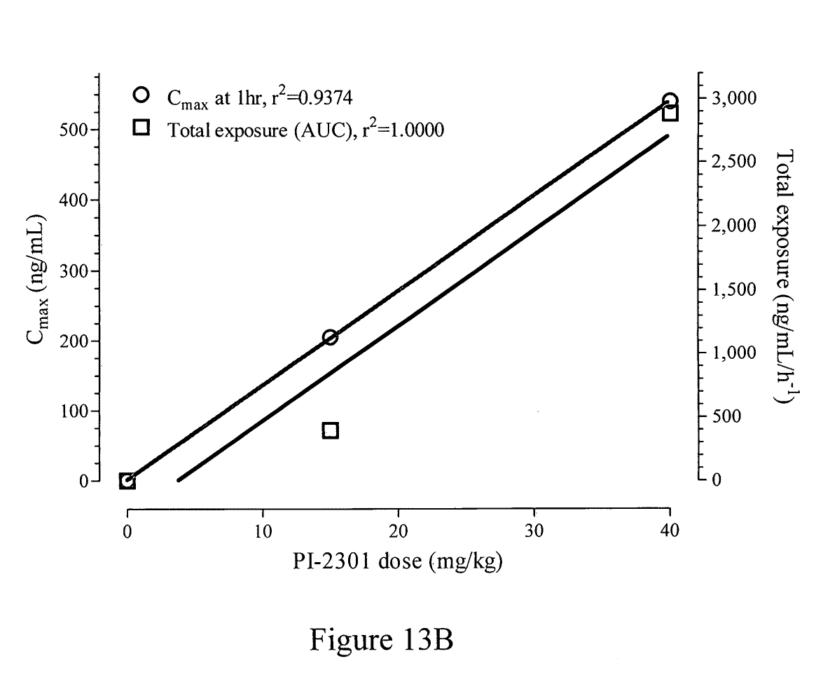

Further, PI-2301's bioavailability for rats was shown to be lower than for mice. FIG. 13A shows the serum concentration of PI-2301 after subcutaneous administration at 15 or 40 mg/kg. Panel B shows the linear correlation between dosed amount and exposure shown by the area under the curve. The maximum exposure was at one hour after administration, and the maximum serum concentration was approximately 40 times lower for the same dosed amount compared to rabbits.

### Example 10

### Detection of PI-2301 in Serum after Administration of the RSP Composition to Humans

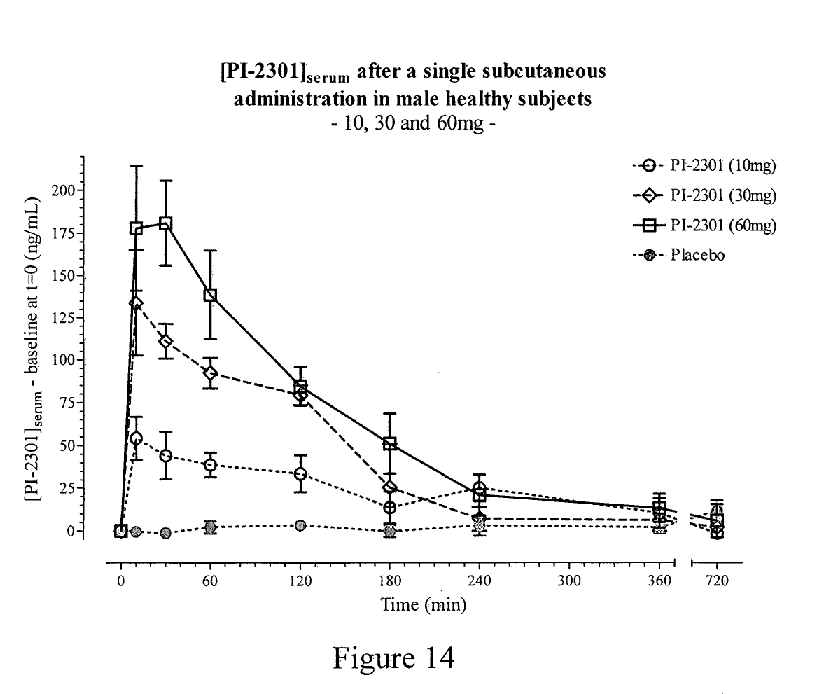

As shown above, the bioavailability differs greatly among different mammalian species. As such, it was necessary to determine the bioavailability in humans instead of extrapolating from animal models. Healthy male human subjects were injected with 0, 10, 30, 60 mg PI-2301 in single dose by subcutaneous route. Samples were taken at 10, 30, 60, 120, 180, 240, 360, or 720 hours, and processed for detection and quantitation of complex peptide mixtures. FIG. 14 shows the results and the table 9 below shows the calculated summary.

TABLE 9
Doses in humans and estimated maximum
serum concentration of PI-2301

| | | | | Predicted | Fold below |
|---|---|---|---|---|---|
| | Total | Dose | Increment | Cmax | Cmax for |
| | Dose (mg) | (mg/kg) | Factor | (μg/mL) | NOAEL |
| | NOAEL | 25.000 | | 3.180 | |
| | MABEL | 0.0500 | | 0.006 | 530 |
| | | | | Predicted | Fold below |
| Cohort | Total | Dose | Increment | Cmax | Cmax for |
| No. | Dose (mg) | (mg/kg) | Factor | (μg/mL) | NOAEL |
| 1 | 0.035 | 0.0005 | | 0.0001 | 50,000 |
| 2 | 0.100 | 0.0014 | 2.86 | 0.0002 | 17,500 |
| 3 | 0.300 | 0.0043 | 3.00 | 0.0005 | 5,833 |
| 4 | 1.000 | 0.0143 | 3.33 | 0.0018 | 1,750 |
| 5 | 3.000 | 0.0429 | 3.00 | 0.0055 | 583 |
| 6 | 10.000 | 0.1429 | 3.33 | 0.0182 | 175 |
| 7 | 30.000 | 0.4286 | 3.00 | 0.0545 | 58 |
| 8 | 60.000 | 0.8571 | 2.00 | 0.1090 | 29 |

### Example 11

### Correlation of Levels of IL1 Receptor Antagonist in Serum with Disease Severity in EAE Mice

IL-1 receptor antagonist (IL1ra) is characteristic of Type II monocytes, and it reduces proinflammatory factors such as TNF-α, IFN-γ, IFN-β, IL-1b, which are induced by IL-1 binding to IL-1 receptors, by competitively binding to IL-1 receptors. Correlation of IL1ra expression with alleviation of the symptoms of multiple sclerosis (MS) through IFNγ treatment has been seen, indicating this cytokine may be a factor mediating symptom improvements by regulation of proinflammatory cytokines. Sciacca et al. (2000) J. Neurovirol., 6 Suppl 2:S33-7; Comabella, M. et al. (2008) J. Neurol., May 20, 2008 e-publication; Dincić E. et al., (2006) J. Neuroimmunol. 177(1-2):146-50.

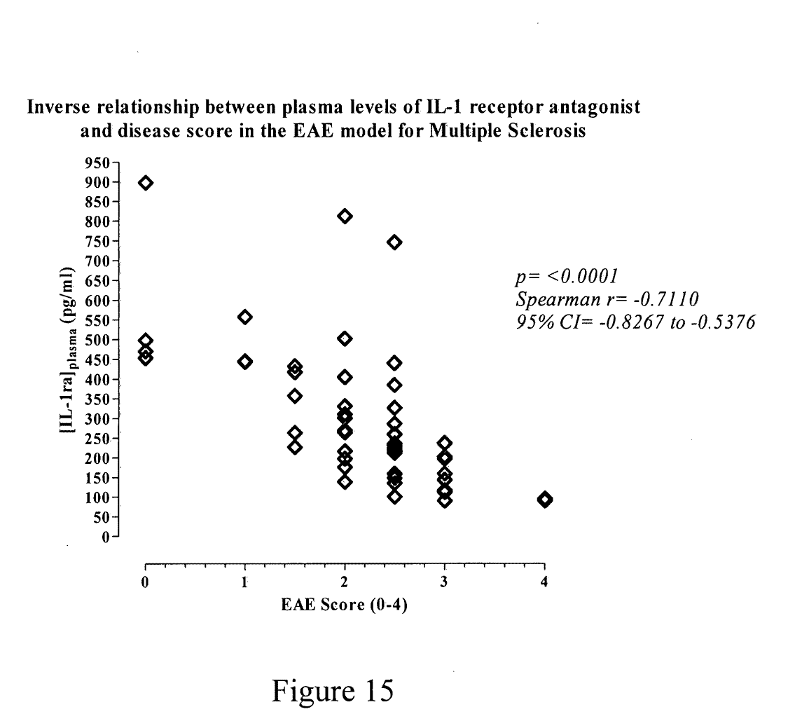

In experimental autoimmune encephalomyelitis (EAE), a mouse model for MS, the plasma levels of IL-1ra is inversely related to the disease score (i.e. severity) (FIG. 15).

An exemplary way of inducing EAE is as follows. Briefly, on day 1, SJL mice are subcutaneously immunized with an emulsion comprised with proteolipid protein (PLP) peptide 139-151 (HSLGKWLGHPDKF) in complete Freund's adjuvant containing heat-killed Mycobacterium tuberculosis H37RA to induce EAE. In addition, this same day, mice are given 200 ng of Pertussis Toxin IP. On day 3 post-immunization, mice are given their second dose of Pertussis toxin IP (200 ng/mouse). The initial signs of disease (paralysis) can usually be observed clearly beginning about 10-13 days after induction. Treated mice are assessed daily for clinical signs of EAE according to an established scale. At score 4, the severity of the disease will require “supportive care” of the animals—food and water supplementation were placed inside the cage to facilitate the feeding needs of the animal(s). Animals that developed life-threatening symptoms are euthanized. Surviving animals are sacrificed about 60 days following EAE induction. The body weight was recorded three times a week. Blood samples are taken at various stages of the disease and levels of IL-1ra are measured.

Data in FIG. 15 show that the disease score is lower for mice having higher plasma levels of IL-1ra.

### Example 12

### Effect of PI-2301 on EAE Mice

EAE mice were treated with different doses of PI-2301 and the disease severity was measured. EAE was induced essentially as described in Example 10. Dosing by PI-2301 at 0 (mannitol, the carrier, only) 2.5 mg/kg daily, or 2.5, 7.5, or 22.5 mg/kg weekly started on Day 8, and continued till Day 52 after induction.

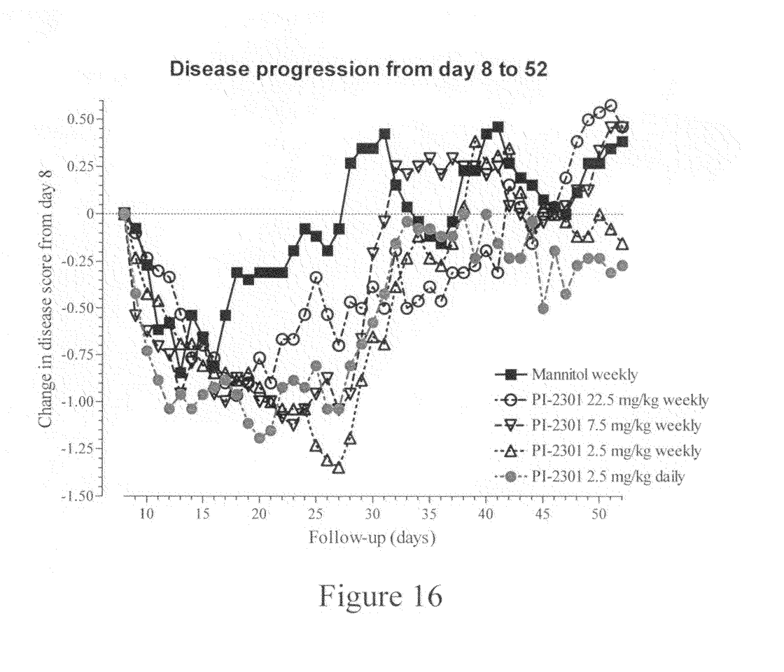

FIG. 16 shows the results. The relative score of disease severity is shown on the Y-axis. Negative change means decrease in the disease severity, i.e. improvement. The mice exhibit a pattern of relapsing/remitting disease, where the symptoms abate for those that were treated with PI-2301 until about 16 to 25 days then worsen again. The mice injected with mannitol only remitted most quickly and experienced generally the worst progress. Those dosed with 2.5 mg/kg weekly fared well, as well as those dosed at the same dose daily. The data show that PI-2301 is effective against EAE.

### Example 13

### Levels of IL-1ra in Serum after Administration of the PI-2301 Composition to Humans

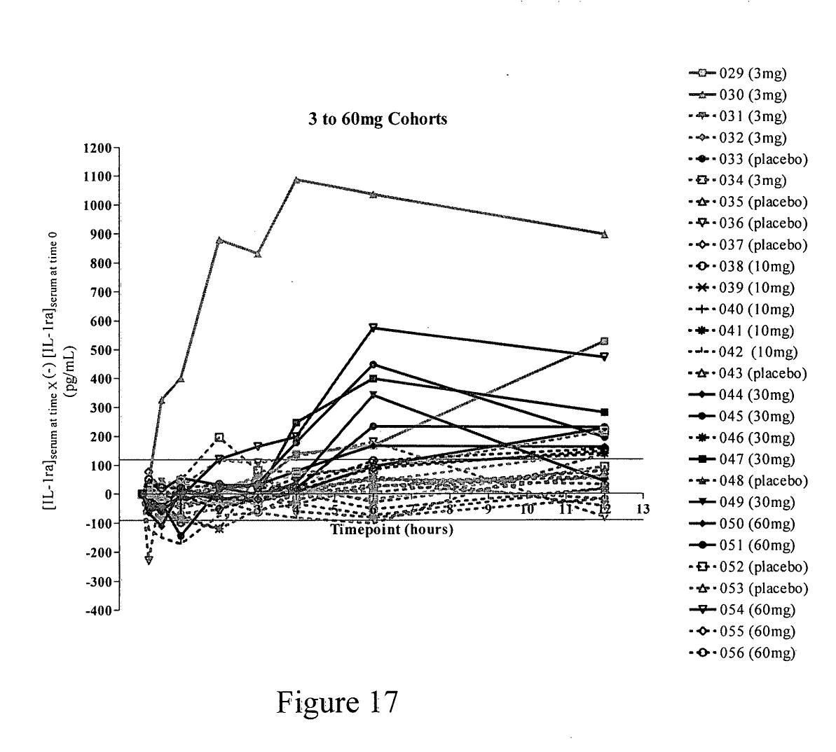

Based on these facts, induction of IL-1ra by PI-2301 was investigated in human subjects. 0, 0.035, 0.1, 0.3, 1, 3, 10, 30, or 60 mg of PI-2301 was administered to healthy adult male and the changes in the plasma levels of IL-1ra were determined at 30 minutes, 1, 2, 3, 4, 6, and 12 hours after administration of PI-2301 using commercially available detection panel. FIG. 17 shows the result of doses from 3 to 60 mg, indicating a general trend of IL-1ra induction.

The data of Examples 11, 12, and 13 together infer that the pharmacodynamics of PI-2301 involve IL-lra, and EAE is a viable model for assessing the effectiveness of PI-2301 and other complex peptide mixtures for the treatment of MS.

### Example 14

### Determination of Correlation between Results in Mice and Human of PI-2301 Dosage and Pharmacodynamic Readout

Experiments are carried out in mice or other experimental animals, and in humans dosing various amounts of PI-2301 at various time intervals. Multiple pharmacodynamic readouts are determined as described in above examples. The results are correlated between human and mice, establishing a coefficient of conversion between mice and human results. Based on such read outs and coefficients, effects of certain dosing regimen in humans are predicted based on the effects in mice.

More concretely, the procedure comprises the following steps.

PI-2301 is prepared starting with Fmoc-Ala-Wang, Fmoc-Lys (Boc)-Wang, Fmoc-Tyr (tBu)-Wang, Fmoc-Phe-Wang. The Fmoc group is cleaved with 20% Piperidine/N-methylpyrrolidone (“NMP”). A mixture of diisopropylethylamine (“DIPEA”)/NMP, Fmoc-Ala-OH, Fmoc-Lys (Boc)-OH, Fmoc-Tyr (tBu)-OH, Fmoc-Phe-OH, O-(Benzotriazol-1-yl)-N,N,N′,N′-tetramethyluronium tetrafluoroborate (“TBTU”)/NMP are used for the coupling/elongation step, wherein the amino acids are present in the mixture in a prescribed ratio. The input molar ratio of Y:F:A:K is 1:1:10:6 to start the peptide synthesis. After the coupling reaction is done, any amino acids that were not incorporated into the peptide chain and residual reagents are washed away and a new batch of the mixture is added for the next cycle of peptide elongation reaction. The elongation process is carried out for a designated number of cycles of reaction and washing, each cycle using a mixture of amino acids in particular ratios. After a desired number of cycles, any further elongation is stopped by acetylating the N-terminus with acetic anhydride, the reactants are filtered, and the peptides are cleaved from the resin support using TFA (trifluoroacetic acid), at the same time removing protective groups of the side chains. The peptides are washed, precipitated by IPE (isopropyl ether), purified and salt exchanged (from TFA to acetic acid salt) by ion exchange column with the mobile phase consisting of ethanol, Ac-52-OH×TFA.

The synthesized PI-2301 is formulated into a sterile, non-pyrogenic aqueous, isotonic, sterile, ready-touse solution at a concentration of 20 mg/mL acceptable for administration to a mammal. The formulation contains mannitol at 42 mg/mL as the only excipient.

The pharmaceutical composition is administered to a subject at the following concentration range: for mice, 2.5, 7.5, and 22.5 mg/kg weekly, and for humans, 1, 3, 10, 15, mg per dose weekly. The subjects are healthy in one cohort and are EAE mouse 8 days after induction of EAE, and patients afflicted with multiple sclerosis for humans in the other. The dose is administered subcutaneously, intramuscularly, transmucosally in bolus manner.

Blood samples are collected every day and plasma or serum is prepared. Concentrations of the YFAK peptides in the plasma or serum samples are determined by ELISA using polyclonal antibodies raised against the YFAK composition. Blood samples are also analyzed for TNFα, IL-6, CXCL1, CXCL2, and IL-12p70 for undesired immune stimulation, and for Il-1ra, CXCL13, and CCL22 for desirable positive changes. Data are analyzed for the amount of PI-2301 administration in relation to a desirable readout, and for correlation between mouse and human data.

Once a desirable readout is determined, the therapeutic subject and other subjects with factors in common with the therapeutic subject is dosed with the desirable dosage at a desirable interval.

We claim:

1. A method of treating or preventing an unwanted immune response in a patient comprising the steps:
a. administering to the patient a pharmaceutical composition comprising a complex peptide mixture at a desirable dosage, wherein such desirable dosage is determined by:
b. administering to an experimental subject a dose of the pharmaceutical composition;
c. removing a tissue sample from said experimental subject;
d. contacting said tissue sample with a means for quantitatively detecting the presence of said complex peptide mixture in said tissue sample;
e. determining the level of said complex peptide mixture in said tissue sample using said means;
f. optionally repeating steps b through f using a different dose; and
g. comparing said level against a predetermined desired level of said complex peptide mixture in said tissue,
wherein the desirable dosage is the dose that results in the predetermined desired level of said complex peptide mixture in said tissue.

2. The method according to claim 1, further comprising the steps of:
i. determining the levels of one or more functional readouts in the tissue sample of said subject by contacting said tissue sample with a means for quantitatively detecting the presence or activity of one or more said functional readouts selected from the group consisting of: hormones, enzymes, serum proteins, cytokines, chemokines, growth factors, immunomodulators, and an effector or regulator of said functional readouts, and
j. comparing said levels of said functional readouts to a predetermined desired level of said functional readout in said tissue,
whereby the desirable dosage is the dose that results in the predetermined desired level of said functional readout in the tissue.

3. The method according to claim 2, wherein said functional readout is selected from the group consisting of: CXCL13, BDNF, CD40, CD40L, IFNγ, IL-1α, IL-1β, IL-1ra, IL-4, IL-5, IL-6, IL-10, IL-12p40, IL-12p70, IL-13, IL-15, IL-16, CCL22, MMP-2, MMP-3, MMP-9, TIMP-1, TNFα, TNFβ, and TNF-RII, CD4, CD8, CD14, CD11b, CD44, CD45RA, CD45RO, CD27, CD123, CD127, CCR5, CCR9, FoxP3, and CCR7, wherein the level of functional readout increases or does not decrease upon administration of said pharmaceutical composition.

4. The method according to claim 2, wherein said functional readout is selected from the group consisting of: Tryptase, IL-2, IL-3, IL-4, IL-6, IL-10, IL-23, IL25, IL-17, IL-27, TNFα, IFNγ, antibodies including IgA, IgE, IgG1, IgG2, IgG3, and IgG4 reactive against the complex peptide mixture, and total IgE serum level, wherein the level of functional readout decreases or does not increase upon administration of said pharmaceutical composition.

5. The method according to claim 1, wherein said means is by immunologic detection.

6. The method according to claim 5, wherein said means is selected from the group consisting of: ELISA, western blot, immunoflow cytometric detection, and radioimmunoassay.

7. The method according to claim 1, whereby the complex peptide mixture is selected from the group consisting of four-amino acid random sequence polymers YEAK, YFAK, VYAK, VEAK, VWAK, and FEAK.

8. The method according to claim 7, wherein the complex peptide mixture is a random sequence copolymer composition comprising YFAK (L-tyrosine, L-phenylalanine, L-alanine and L-lysine) in an output molar ratio of about 1.0:1.2:XA:6.0 respectively, synthesized by solid phase chemistry, and has a length of at least 35 amino acids wherein XA=20.0 to 30.0.

9. The method according to claim 1, wherein the unwanted immune response is selected from the group consisting of multiple sclerosis, rheumatoid arthritis, and Crohn's disease.

10. The method of claim 1, wherein the complex peptide mixture is delivered in a sustained release formulation.

11. The method according to claim 1, wherein the route of administration is selected from the group consisting of subcutaneous injection, peritoneal injection, intravenous injection, intramuscular injection, buccal administration, transmucosal administration, and transdermal administration.

12. The method according to claim 1, wherein said tissue sample is blood or bodily fluid.

13. The method according to claim 1, wherein said readout is IL-1 receptor antagonist.

14. A method of preventing or treating an unwanted immune response in a patient by administering a composition comprising a complex peptide mixture at a desirable dosage, comprising the steps of:
a. preparing a complex peptide mixture by solid phase or solution phase peptide synthesis;
b. preparing a pharmaceutically acceptable formulation of said complex peptide mixture;
c. administering said complex peptide mixture to the patient;
d. quantitatively detecting a level of a functional readout from the patient that correlates with bioavailability of said complex peptide mixture in said tissue sample;
e. optionally repeating steps (b) through (c) using a different dose of said complex peptide mixture; and
f. comparing said level against a predetermined desired level of said functional readout,
wherein the desirable dosage is the dose that results in the predetermined desired level of said functional readout.

15. The method according to claim 14, wherein step (c) is repeated after certain time intervals to determine the time-course of bioavailability after administration to determine desired time interval of administering said complex peptide mixture.

16. A method of improving the manufacturing process of a composition comprising a complex peptide mixture, such method comprising preparing a complex peptide mixture according to a protocol, further preparing a composition comprising said complex peptide mixture, determining the bioavailable amount of complex peptide mixture in said composition by detecting the level or degree of functional readout, comparing such readout against a standard, and adjusting the protocol or the step of preparing the composition to obtain a desired bioavailability.

17. A method of determining bioavailability of a complex peptide mixture in a mammal, comprising the steps of:
a. administering to an experimental subject a dose of the pharmaceutical composition;
b. removing a tissue sample from said experimental subject;
c. contacting said tissue sample with a means for quantitatively detecting the presence of said complex peptide mixture in said tissue sample; and
determining the level of said complex peptide mixture in said tissue sample using said means.
